(19) United States
(12) Patent Application Publication
(10) Pub. No.: US 2022/0226241 A1
(43) Pub. Date: Jul. 21, 2022

(54) STABLE ORGANIC CANNABINOID OIL BLEND FORMULATIONS

(71) Applicant: Therabody, Inc., Los Angeles, CA (US)

(72) Inventor: Timothy Drennan, Denver, CO (US)

(21) Appl. No.: 17/344,415

(22) Filed: Jun. 10, 2021

Related U.S. Application Data
(60) Provisional application No. 63/139,579, filed on Jan. 20, 2021.

(51) Int. Cl. A61K 9/12; A61K 47/26; A61K 47/36; A61K 47/10; A61K 47/46; A61K 9/06; A61K 47/14; A61K 31/05; A61K 47/44

(52) U.S. Cl. CPC A61K 9/122; A61K 47/26; A61K 47/36; A61K 47/10; A61K 47/44; A61K 9/06; A61K 47/14; A61K 31/05; A61K 47/46

(57) ABSTRACT

The present application is directed to thermally stable cannabinoid oil-containing topical formulations, such as lotions and balms. The formulations include sodium alginate and comprise greater than 95% USDA certified organic ingredients. These formulations may be used for dermal or transdermal application useful in the treatment of, e.g., pain. Also disclosed are methods of applying such formulations.

### CROSS REFERENCE

This application claims the benefit of U.S. Provisional Application No. 63/139,579, filed Jan. 20, 2021 all of which is herein incorporated by reference in its entirety.

### FIELD OF THE DISCLOSURE

The present disclosure relates to cannabinoid oil-comprising lotions and balms that comprise greater than 95% USDA certified organic ingredients, comprise sodium alginate, and comprise an effective ratio of ingredients in water, oil, and preservative phases. These formulations are for topical application.

### BACKGROUND

Cannabis is one of the most widely used herbs for medicinal purposes. Medical cannabis is used for treating and alleviating symptoms associated with a growing number of indications, including pain, anorexia, asthma, glaucoma, arthritis, spasms, anxiety, and substance withdrawal. Many other illnesses are emerging as potential cannabis-responsive indications. Cannabidiol (CBD) is a phytocannabinoid, one of the most plentiful of the numerous cannabinoids derived from Cannabis plants. CBD is non-psychotropic, but has biochemical and pharmacological features that make it relevant for numerous therapeutic applications. Existing studies have explored the use of CBD in treating, for example, neurological disorders, seizures, anxiety, diabetes, nausea, arthritis and even cancer. CBD has also been used for thousands of years to treat various forms of pain.

Lotions and other topical compositions comprising CBD provide a means by which to improve symptoms of various conditions via application to the skin. However, few if any commercially available CBD lotions are made with a high proportion of organic ingredients, because of the many difficulties in obtaining a highly organic product with the desired characteristics for a topical composition, such as thermostability and consistency.

There is an ongoing, unmet need for highly organic cannabinoid-comprising lotions and balms.

### BRIEF SUMMARY

Provided herein is an oil-in-water emulsion composition comprising: (a) 40-60% water by weight; (b) 11%-30% carrier oil by weight; (c) at least 1% cannabinoid by weight; (d) a foaming agent; and (e) an emulsion stabilizer. In embodiments, percent content is by weight of the topical oil-in-water emulsion composition. In embodiments, the oil-in-water composition is thermostable.

In embodiments, an essential oil is selected from the group consisting of: rose oil, menthol, tea tree oil, lavender, camphor, arnica, black pepper oil, turmeric, peppermint, eucalyptus oil, lemon balm, chamomile oil, clove oil, rose geranium oil, and sea buckthorn oil.

In embodiments, the foaming agent comprises saponin.

In embodiments, the foaming agent comprises 0.1-5.0% saponin by weight.

In embodiments, the emulsion stabilizer comprises sodium alginate or sodium alginate and Guar Gum.

In embodiments, the emulsion stabilizer comprises 0.1-5.0% sodium alginate by weight.

In embodiments, the cannabinoid is comprised in a Therapeutic Oil Blend.

In embodiments, the cannabinoid comprises cannabidiol.

In embodiments, the oil-in-water emulsion comprises ethanol.

In embodiments, the oil-in-water emulsion comprises 10-30% ethanol by weight.

In embodiments, the oil-in-water emulsion composition comprises glycerin by weight.

In embodiments, the oil-in-water emulsion composition comprises 1-10% glycerin by weight.

In embodiments, the oil-in-water emulsion composition has reduced or no: separation, discoloration, or change in consistency at 37° C. for a period of three months.

In embodiments, thermostability is determined by evaluating at least one of: separation, discoloration, or consistency, of the oil-in-water emulsion composition at 45° C. for a period of three months.

In embodiments, the oil-in-water emulsion is composed of at least 95% USDA certified organic ingredients.

In embodiments, the oil-in-water emulsion exhibits a viscosity of about 3,000-7,000 cps.

Provided herein is also an oil-in-water emulsion composition, that comprises: (a) Water; (b) Ethanol; (c) Olive Oil; (d) Sunflower Oil; (e) Hemp Seed Oil; (f) Glycerin; (g) Lavender Oil; (h) Menthol; (i) Camphor; (j) Eucalyptus Oil; (k) Arnica Oil; (l) Black Pepper Oil; (m) Cannabinoid; (n) Saponin; (o) Sodium Alginate; (p) Turmeric Oil; (q) Sea Buckthorn Oil; and (r) Guar Gum.

In embodiments, the oil-in-water emulsion composition comprises: (a) 40-60% Water by weight; (b) 10-30% Ethanol by weight; (c) 1-10% Olive Oil by weight; (d) 1-10% Sunflower Oil by weight; (e) 0.1-2.0% Hemp Seed Oil by weight; (f) 1-10% Glycerin by weight; (g) 0.1-5.0% Lavender Oil by weight; (h) 0.1-5.0% Menthol by weight; (i) 0.1-5.0% Camphor by weight; (j) 0.1-5.0% Eucalyptus Oil by weight; (k) 0.1-5.0% Arnica Oil by weight; (l) 0.1-5.0% Black Pepper Oil by weight; (m) 0.1-5.0% Cannabinoid by weight; (n) 0.1-5.0% Saponin by weight; (o) 0.1-5.0% Sodium Alginate by weight; (p) 0.1-5.0% Turmeric Oil by weight; (q) 0.1-1.0% Sea Buckthorn Oil by weight; and (r) 0.1-1.0% Guar Gum by weight. In embodiments, percent content is by weight of the oil-in-water emulsion composition.

In embodiments, the oil-in-water emulsion composition comprises about: (a) 51% Water by weight; (b) 20% Ethanol by weight; (c) 6% Olive Oil by weight; (d) 6% Sunflower Oil by weight; (e) 1% Hemp Seed Oil by weight; (f) 5% Glycerin by weight; (g) 1.5% Lavender Oil by weight; (h) 1.5% Menthol by weight; (i) 1.5% Camphor by weight; (j) 1.5% Eucalyptus Oil by weight; (k) 1% Arnica Oil by weight; (l) 1% Black Pepper Oil by weight; (m) 1% Cannabinoid by weight; (n) 1% Saponin by weight; (o) 1% Sodium Alginate by weight; (p) 1% Turmeric Oil by weight; (q) 0.3% Sea Buckthorn Oil by weight; and (r) 0.3% Guar Gum. In embodiments, percent content is by weight of the oil-in-water emulsion composition.

Provided is also an oil-in-water emulsion composition, that comprises: (a) Water; (b) Ethanol; (c) Olive Oil; (d) Sunflower Oil; (e) Hemp Seed Oil; (f) Glycerin; (g) Tea Tree Oil; (h) Arnica Oil; (i) Lemon Balm Oil; (j) Eucalyptus Oil; (k) Rose Geranium Oil; (l) Cannabinoid; (m) Saponin; (n) Chamomile Oil; (o) Sodium Alginate; (p) Guar Gum; (q) Sea Buckthorn Oil; and (r) Peppermint Oil.

In embodiments, the oil-in-water emulsion composition comprises: (a) 40-50% Water by weight; (b) 10-30% Ethanol by weight; (c) 1-10% Olive Oil by weight; (d) 1-10% Sunflower Oil by weight; (e) 1-10% Hemp Seed Oil by weight; (f) 1-10% Glycerin by weight; (g) 0.1-5% Tea Tree Oil by weight; (h) 0.1-5% Arnica Oil by weight; (i) 0.1-5% Lemon Balm Oil by weight; (j) 0.1-5% Eucalyptus Oil by weight; (k) 0.1-5% Rose Geranium Oil by weight; (l) 0.1-5% Cannabinoid by weight; (m) 0.1-5% Saponin by weight; (n) 0.1-5% Chamomile Oil by weight; (o) 0.1-5% Sodium Alginate by weight; (p) 0.1-1% Guar Gum by weight; (q) 0.1-1% Sea Buckthorn Oil by weight; and (r) 0.1-5% Peppermint Oil by weight. In embodiments, percent content is by weight of the oil-in-water emulsion composition. In embodiments, the oil-in-water emulsion composition comprises about: (a) 43% Water by weight; (b) 20% Ethanol by weight; (c) 6% Olive Oil by weight; (d) 6% Sunflower Oil by weight; (e) 6% Hemp Seed Oil by weight; (f) 5% Glycerin by weight; (g) 1% Tea Tree Oil by weight; (h) 1% Arnica Oil by weight; (i) 3% Lemon Balm Oil by weight; (j) 2% Eucalyptus Oil by weight; (k) 1% Rose Geranium Oil by weight; (l) 1% Cannabinoid by weight; (m) 1% Saponin by weight; (n) 1% Chamomile Oil by weight; (o) 1% Sodium Alginate by weight; (p) 0.3% Guar Gum by weight; (q) 0.3% Sea Buckthorn Oil by weight; and (r) 3% Peppermint Oil by weight. In embodiments, percent content is by weight of the oil-in-water emulsion composition.

Provided is also a balm composition, that comprises: (a) Shea Nut Butter; (b) Beeswax; (c) MCT Oil; (d) Turmeric Oil; (e) Cannabinoid; (f) Arnica Oil; (g) Menthol; (h) Camphor; and (i) Eucalyptus Oil.

In embodiments, the balm composition comprises: (a) 10-40% Shea Nut Butter by weight; (b) 20-50% Beeswax by weight; (c) 20-30% MCT Oil by weight; (d) 1-10% Turmeric Oil by weight; (e) 1-10% Cannabidiol by weight; (f) 1-5% Arnica Oil by weight; (g) 0.1-5.0% Menthol by weight; (h) 0.1-5.0% Camphor by weight; and (i) 0.1-5.0% Eucalyptus Oil by weight. In embodiments, percent content is by weight of the balm composition. In embodiments, the balm composition comprises about: (a) 30% Shea Nut Butter by weight; (b) 30% Beeswax by weight; (c) 27% MCT Oil by weight; (d) 4% Turmeric Oil by weight; (e) 4% Cannabidiol by weight; (f) 2% Arnica Oil by weight; (g) 1% Menthol by weight; (h) 1% Camphor by weight; and (i) 1% Eucalyptus Oil by weight. In embodiments, percent content is by weight of the balm composition.

In one aspect, the present disclosure provides a topical oil-in-water emulsion composition comprising: a) Water; b) Ethanol; c) Olive Oil; d) Sunflower Oil; e) Hemp Seed Oil; f) Glycerin; g) Lavender Oil; h) Menthol; i) Camphor; j) Eucalyptus Oil; k) Arnica Oil; l) Black Pepper Oil; m) CBD Extract; n) Saponin; o) Sodium Alginate; p) Turmeric Oil; q) Sea Buckthorn Oil; and r) Guar Gum.

In some embodiments, the composition comprises about: a) 40-60% Water; b) 10-30% Ethanol; c) 1-10% Olive Oil; d) 1-10% Sunflower Oil; e) 0.1-2.0% Hemp Seed Oil; f) 1-10% Glycerin; g) 0.1-5.0% Lavender Oil; h) 0.1-5.0% Menthol; i) 0.1-5.0% Camphor; j) 0.1-5.0% Eucalyptus Oil; k) 0.1-5.0% Arnica Oil; l) 0.1-5.0% Black Pepper Oil; m) 0.1-5.0% CBD Extract; n) 0.1-5.0% Saponin; o) 0.1-5.0% Sodium Alginate; p) 0.1-5.0% Turmeric Oil; q) 0.1-1.0% Sea Buckthorn Oil; and r) 0.1-1.0% Guar Gum, wherein the percent content is weight of ingredient by weight of the composition.

In some embodiments, the composition comprises about: a) 51% Water; b) 20% Ethanol; c) 6% Olive Oil; d) 6% Sunflower Oil; e) 1% Hemp Seed Oil; f) 5% Glycerin; g) 1.5% Lavender Oil; h) 1.5% Menthol; i) 1.5% Camphor; j) 1.5% Eucalyptus Oil; k) 1% Arnica Oil; l) 1% Black Pepper Oil; m) 1% CBD Extract; n) 1% Saponin; o) 1% Sodium Alginate; p) 1% Turmeric Oil; q) 0.3% Sea Buckthorn Oil; and r) 0.3% Guar Gum, wherein the percent content is weight of ingredient by weight of the composition.

In some embodiments, the composition comprises per two ounce volume about: a) 20-40 g Water; b) 5-20 g Ethanol; c) 1-5 g Olive Oil; d) 1-5 g Sunflower Oil; e) 0.1-0.5 g Hemp Seed Oil; f) 1-5 g Glycerin; g) 0.1-2 g Lavender Oil; h) 0.1-2 g Menthol; i) 0.1-2 g Camphor; j) 0.1-2 g Eucalyptus Oil; k) 0.1-2 g Arnica Oil; l) 0.1-2 g Black Pepper Oil; m) 0.1-2 g CBD Extract; n) 0.1-2 g Saponin; o) 0.1-2 g Sodium Alginate; p) 0.1-2 g Turmeric Oil; q) 0.1-2 g Sea Buckthorn Oil; and r) 0.1-2 g Guar Gum.

In some embodiments, the composition comprises per two ounce volume about: a) 29 g Water; b) 11 g Ethanol; c) 3 g Olive Oil; d) 3 g Sunflower Oil; e) 0.3 g Hemp Seed Oil; f) 2.5 g Glycerin; g) 0.9 g Lavender Oil; h) 0.9 g Menthol; i) 0.9 g Camphor; j) 0.9 g Eucalyptus Oil; k) 0.6 g Arnica Oil; l) 0.6 g Black Pepper Oil; m) 0.6 g CBD Extract; n) 0.6 g Saponin; o) 0.6 g Sodium Alginate; p) 0.6 g Turmeric Oil; q) 0.2 g Sea Buckthorn Oil; and r) 0.2 g Guar Gum.

In one aspect, the present disclosure provides a topical oil-in-water emulsion composition comprising: a) Water; b) Ethanol; c) Olive Oil; d) Sunflower Oil; e) Hemp Seed Oil; f) Glycerin; g) Tea Tree Oil; h) Arnica Oil; i) Lemon Balm Extract; j) Eucalyptus Oil; k) Rose Geranium Oil; l) CBD Extract; m) Saponin; n) Chamomile Extract; o) Sodium Alginate; p) Guar Gum; q) Sea Buckthorn Oil; and r) Peppermint Oil.

In some embodiments, the composition comprises about: a) 40-50% Water; b) 10-30% Ethanol; c) 1-10% Olive Oil; d) 1-10% Sunflower Oil; e) 1-10% Hemp Seed Oil; f) 1-10% Glycerin; g) 0.1-5% Tea Tree Oil; h) 0.1-5% Arnica Oil; i) 0.1-5% Lemon Balm Extract; j) 0.1-5% Eucalyptus Oil; k) 0.1-5% Rose Geranium Oil; l) 0.1-5% CBD Extract; m) 0.1-5% Saponin; n) 0.1-5% Chamomile Extract; o) 0.1-5% Sodium Alginate; p) 0.1-1% Guar Gum; q) 0.1-1% Sea Buckthorn Oil; and r) 0.1-5% Peppermint Oil, wherein the percent content is weight of ingredient by weight of the composition.

In some embodiments, the composition comprises about: a) 43% Water; b) 20% Ethanol; c) 6% Olive Oil; d) 6% Sunflower Oil; e) 6% Hemp Seed Oil; f) 5% Glycerin; g) 1% Tea Tree Oil; h) 1% Arnica Oil; i) 3% Lemon Balm Extract; j) 2% Eucalyptus Oil; k) 1% Rose Geranium Oil; l) 1% CBD Extract; m) 1% Saponin; n) 1% Chamomile Extract; o) 1% Sodium Alginate; p) 0.3% Guar Gum; q) 0.3% Sea Buckthorn Oil; and r) 3% Peppermint Oil, wherein the percent content is weight of ingredient by weight of the composition.

In some embodiments, the composition comprises per two ounce volume about: a) 20-30 g Water; b) 5-15 g Ethanol; c) 1-5 g Olive Oil; d) 1-5 g Sunflower Oil; e) 1-5 g Hemp Seed Oil; f) 1-5 g Glycerin; g) 0.1-2.0 g Tea Tree Oil; h) 0.1-2.0 g Arnica Oil; i) 0.1-5.0 g Lemon Balm Extract; j) 0.1-5.0 g Eucalyptus Oil; k) 0.1-2.0 g Rose Geranium Oil; l) 0.1-2.0 g CBD Extract; m) 0.1-2.0 g Saponin; n) 0.1-2.0 g Chamomile Extract; o) 0.1-2.0 g Sodium Alginate; p) 0.1-1.0 g Guar Gum; q) 0.1-1.0 g Sea Buckthorn Oil; and r) 1-5 g Peppermint Oil.

In some embodiments, the composition comprises per two ounce volume about: a) 24 g Water; b) 11 g Ethanol; c) 3 g Olive Oil; d) 3 g Sunflower Oil; e) 3 g Hemp Seed Oil; f) 3 g Glycerin; g) 0.6 g Tea Tree Oil; h) 0.6 g Arnica Oil; i) 2 g Lemon Balm Extract; j) 1 g Eucalyptus Oil; k) 0.6 g Rose Geranium Oil; l) 0.6 g CBD Extract; m) 0.6 g Saponin; n) 0.6 g Chamomile Extract; o) 0.6 g Sodium Alginate; p) 0.2 g Guar Gum; q) 0.2 g Sea Buckthorn Oil; and r) 2 g Peppermint Oil.

In one aspect, the present disclosure provides a balm composition comprising: a) Shea Nut Butter; b) Beeswax; c) MCT Oil; d) Turmeric Oil; e) CBD Extract; f) Arnica Oil; g) Menthol; h) Camphor; and i) Eucalyptus Oil.

In some embodiments, the composition comprises about: a) 10-40% Shea Nut Butter; b) 20-50% Beeswax; c) 20-30% MCT Oil; d) 1-10% Turmeric Oil; e) 1-10% CBD Extract; f) 1-5% Arnica Oil; g) 0.1-5.0% Menthol; h) 0.1-5.0% Camphor; and i) 0.1-5.0% Eucalyptus Oil, wherein the percent content is weight of ingredient by weight of the composition.

In some embodiments, the composition comprises about: a) 30% Shea Nut Butter; b) 30% Beeswax; c) 27% MCT Oil; d) 4% Turmeric Oil; e) 4% CBD Extract; f) 2% Arnica Oil; g) 1% Menthol; h) 1% Camphor; and i) 1% Eucalyptus Oil, wherein the percent content is weight of ingredient by weight of the composition.

In some embodiments, the composition comprises per one ounce about: a) 5-15 g Shea Nut Butter; b) 5-15 g Beeswax; c) 5-10 g MCT Oil; d) 0.1-5 g Turmeric Oil; e) 0.1-5 g CBD Extract; f) 0.1-5 g Arnica Oil; g) 0.1-2 g Menthol; h) 0.1-2 g Camphor; and i) 0.1-2 g Eucalyptus Oil.

In some embodiments, the composition comprises per one ounce about: a) 9 g Shea Nut Butter; b) 8 g Beeswax; c) 7.5 g MCT Oil; d) 1 g Turmeric Oil; e) 1 g CBD Extract; f) 0.5 g Arnica Oil; g) 0.3 g Menthol; h) 0.3 g Camphor; and i) 0.3 g Eucalyptus Oil.

### BRIEF DESCRIPTION OF THE SEVERAL VIEWS OF THE DRAWINGS

FIGS. 1A-E shows Lotions A, B, C, and D described in Examples 11 and 12. FIG. 1A shows Lotion A; FIG. 1B shows Lotion B; FIG. 1C shows Lotion C; and FIG. 1D shows Lotion D; and FIG. 1E shows a side-by-side comparison of all four lotions. In FIG. 1E, Lotion A is in the upper left, Lotion B is in the upper right, Lotion C is in the lower left, and Lotion D is in the lower right.

### DETAILED DESCRIPTION

### Definitions

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

An “analgesic,” as used herein, refers to a substance that induces analgesia, or relief from pain. Analgesics act in various ways on the peripheral and central nervous systems to prevent, block, reduce, or eliminate the sensation of pain.

As used herein, an “anesthetic” refers to a substance that induces anesthesia, or the temporary loss of sensation or awareness. Anesthetics temporarily affect, and in some instances completely eliminate, sensation, not exclusively limited to the sensation of pain.

As used herein, the term “bioavailability” refers to the proportion of a drug or other substance that is capable of being absorbed and used by the body, and is therefore able to have an active effect.

As used herein, the term “cannabinoid” means an endocannabinoid receptor ligand, and/or molecules explicitly defined as cannabinoids in this specifiation. Cannabinoids include phytocannabinoids, which are cannabinoids that are naturally produced by plants of genus Cannabis, including the acidic and decarboxylated acid forms of the naturally-occurring plant-derived cannabinoids, and also includes cannabinoids produced from synthetic and biosynthetic methods that are identical to naturally-occurring plant-derived cannabinoids. Additional information about cannabinoids is provided in later sections of this application.

The term “cannabis” refers to a genus of flowering plants. Plants of genus cannabis include several species, including Cannabis sativa, Cannabis indica, and Cannabis ruderalis. There is a long history of cultivating plants of genus cannabis for hemp fibers, seeds and seed oils, medicinal purposes, and recreational activities.

The term “cannabis extract” as used herein refers to one or more plant extracts from the cannabis plant. A cannabis extract may contain, in addition to one or more cannabinoids, one or more non-cannabinoid components that are co-extracted with the cannabinoids from the plant material. Their respective ranges in weight will vary according to the starting plant material and the extraction methodology used. Cannabinoid-containing plant extracts may be obtained by various means of extraction of cannabis plant material. Such means include but are not limited to supercritical or subcritical extraction with CO₂, extraction with hot or cold gas and extraction with solvents.

As used herein, “cavitation” refers to the formation of an empty space within a substance, e.g., the formation of bubbles within a liquid. During sonication, cycles of pressure form thousands of microscopic vacuum bubbles in the solution. The bubbles collapse into the solution in a process known as cavitation.

The verb “comprise,” as is used in this description and in the claims and its conjugations, is used in its non-limiting sense to mean that items following the word are included, but items not specifically mentioned are not excluded.

The term “emulsifier” as used herein refers to amphiphilic molecules that are surface-active agents and that stabilize emulsions by reducing the interfacial tension.

The term “essential oils” as used herein refers to a concentrated liquid containing volatile aroma compounds. Essential oils may be plant-derived, synthetically produced, or biosynthetically produced. Essential oils are also known as volatile oils, ethereal oils, aetherolea, or the oil of the plant from which they were extracted. The term “essential oil” may also refer to natural plant oil typically obtained by distillation and having a chemical composition and organoleptic properties (e.g., fragrance) characteristic of the plant or other source from which it is extracted.

As used herein, the term “fat” refers to saturated, mono-unsaturated and poly-unsaturated fatty acid. Fatty acids are usually present in the form of esters (e.g. mono-/di-/triglycerides).

As used herein the term “oil” is used as a generic term for lipids, fats, or any mixture thereof. A “carrier oil” is an oil used to dilute, distribute, and generally carry another substance with which it is mixed. Common carrier oils include plant-derived oils, e.g., vegetable oils and nut oils.

“Hemp oil” refers to a botanical extract comprised of extracted hemp flowers, e.g., CO₂-extracted with an ethanol co-solvent. Post refinement removes most lipids and waxes in the oil. The oil is high in CBD, CBG, CBC with other minor cannabinoids. Terpenes are less than 1% total make up. This is distinct from Hemp seed oil, which is oil extracted from hemp seeds. In contrast with hemp oil, hemp seed oil does not comprise high levels of cannabinoids.

The term “nano-penetrative” as used herein refers to a composition, e.g., a therapeutic oil blend, that has an average particle size of less than 0.1 microns, thereby increasing bioavailability through a dermal membrane or mucous membrane. In some embodiments, all of the constituents of the composition have an average particle size of less than 0.1 microns. In some embodiments, some key ingredients of the composition, e.g., the cannabinoids and/or the terpenes, are suspended or contained within phases having an average particle size of less than 0.1 microns.

The term “pharmaceutical composition” as used herein refers to a composition that is pharmaceutically acceptable. The term “pharmaceutically acceptable” as used herein refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio.

The term “excipient” as used herein refers to a pharmaceutically acceptable ingredient, which is commonly used in the pharmaceutical technology for preparing a granulate, solid or liquid oral dosage formulation.

The term “cosmetic composition” is intended to mean a substance or a preparation intended to be brought into contact with the various superficial parts of the body, in particular the epidermis, the body-hair and head-hair systems, the nails, the lips and the oral mucous membranes.

A “surfactant” as used herein refers to a substance that reduces the surface tension of a liquid in which it is dissolved.

The term “terpene” as used herein also covers terpenoids. Terpenes are lipophilic compounds, volatile and liquid at room temperature and are used herein in this disclosure as cannabinoid solubilizing agents. Terpenes are major secondary metabolites of cannabis and are responsible for the odor and flavor of various cannabis strains. Cannabis strains and hemp strains produce many terpenes as secondary metabolites. Terpenes are synthesized from terpene unit into mono-terpenes, sesqui-terpenes, and di-terpenes that are lipophilic, volatile and insoluble in water and are cyclic or bicyclic or not cyclic and may have alcohol, aldehyde or ketone chemical moiety. The term “terpene” further relates to essential oils. The term “terpene” does not include fats and/or lipids.

“Treatment,” “treating,” “palliating” and “ameliorating,” as used herein, are used interchangeably. These terms refer to an approach for obtaining beneficial or desired results including but not limited to therapeutic benefit and/or a prophylactic benefit. Therapeutic benefit can be eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. Treating a disorder include reducing, alleviating, abating, ameliorating, relieving, or lessening a symptom associated with a disorder. In some embodiments, the term includes, but is not limited to, alleviation or amelioration of one or more symptoms or parameters associated with a disease, such as improvement in parameters as assessed various rating scales, tests or indices.

As used herein, the term “vasodilation” refers to the dilation of blood vessels. Vasodilation may be naturally or artificially induced, e.g., through the application of vasodilating compounds. Vasodilation may be accompanied by other physiological effects, such as lowering of blood pressure.

The term “vitamin E” refers to a group of compounds that include both tocopherols and tocotrienols including, but not limited to a-tocopherol, β-tocopherol, γ-tocopherol, δ-tocopherol, a-tocotrienol, β-tocotrienol, γ-tocotrienol, δ-tocotrienol, salts thereof, and combinations thereof. Vitamin E can be obtained from sources including, but not limited, to soybeans, sunflowers, and combinations thereof.

“Thermostable” as used herein refers to a topical composition that does not separate, discolor, or change in consistency within a specific temperature range for some period of time, or which exhibits reduced separation, discoloration, or changes in consistency compared to a non-thermostable composition. In some embodiments, the composition is considered thermostable if it can be stored at room temperature (e.g., less than 80° F.) for a period of at least six months without noticeably separating, discoloring, or changing in consistency (i.e., no separation, discoloration or change in consistency ascertainable by the naked/eye or touch of a user). In some embodiments, thermostability is assessed by exposing the composition to extreme temperatures for a shorter period of time to predict long-term stability within normal temperature ranges. In some embodiments, the composition is considered thermostable if it can be stored at 37° C. for a period of three months without noticeable separation, discoloration, or changes in consistency. In some embodiments, the composition is considered thermostable if it can be stored at 45° C. for a period of three months without noticeable separation, discoloration, or changes in consistency.

The term “noticeable,” “noticeably,” and the like refers to the average user's ability to discern the described change (e.g., change in consistency, discoloration, or consistency.

The “water phase” of the compositions of the present disclosure refers to the water and water-soluble components of the composition. The water phase does not include alcohols, even if they are soluble in water.

The “oil phase” of the compositions of the present disclosure refers to the oil and oil-soluble components of the composition.

The “preservative phase” of the compositions of the present disclosure refers to the preservative components of the composition. In some embodiments, the preservative phase comprises the alcohol and alcohol-based components of the composition. This phase may also be referred to as the “alcohol phase” or the “ethanol phase”.

An “effective water:oil:preservative ratio” as used herein describes a ratio of the three phases of the composition that allows for certain desirable characteristics of the composition. In some embodiments, the desirable characteristics are consistency and thermostability.

### Organic Products

Embodiments of the present disclosure are directed to compositions for topical application, e.g., lotions and balms, comprising at least 95% organic ingredients. In the United States, the Agricultural Marketing Service of the U.S. Department of Agriculture (USDA) oversees the National Organic Program (NOP). The NOP regulations include a definition of “organic” and provide for certification that agricultural ingredients have been produced under conditions that would meet the definition. They also include labeling standards based on the percentage of organic ingredients in a product.

The NOP applies the term “Organic” to products containing at least 95 percent organically produced ingredients (excluding water and salt). Remaining product ingredients must consist of nonagricultural substances approved on the National List or non-organically produced agricultural products that are not commercially available in organic form. See ams.usda.gov/rules-regulations/organic/national-list). Products meeting these requirements may display the USDA Organic Seal and must display the certifying agent's name and address.

There are numerous difficulties in meeting the stringent definition of an organic product in the beauty and cosmetics industries. In particular, numerous non-organic chemical constituents of typical products are prohibited from inclusion in organic products or shift the percentage of organic ingredients to less than 95%, thereby rendering the product not “organic.” Without the aid of synthetic cross-polymers, it is very challenging to obtain products having the requisite thermostability and consistency. To the best of Applicant's knowledge, the only commercially available lotion meeting this strict USDA organic standard is the Peppermint Organic Hand and Body Lotion produced by Dr. Bronner's ALL-ONE!® brand. Dr. Bronner's formulation however is limited in its applications for other cosmetic products due to its low viscosity. Specifically, Dr. Bronner's lotion makes use of the ingredient “xanthan gum” for thickening, which only achieves a viscosity of around 700-800 cps. Most consumers prefer firmer lotions, and typically demand viscosities in the 3,000-7,000 cps range.

Given the difficulties of creating organic beauty products, as demonstrated by the scarcity of commercially available products meeting these stringent requirements, the inventor of the present application was surprisingly able to develop organic lotions and balms overcoming the problems of thermostability and consistency associated with wholly organic products. In some embodiments, the present compositions are stable at room temperature for a period of at least six months. In embodiments, stability (e.g. thermostability) is determined by evaluating at least one of: separation, discoloration, or consistency, of the lotion composition at 45° C. for a period of three months. In some embodiments, the lotion compositions of the present disclosure have a viscosity in the range of 5000-6500 cps.

The present compositions comprise greater than 95% organic components. Other ingredients suitable for inclusion in the compositions of the disclosure are listed in The National List of Allowed and Prohibited Substances, which may be found at ams.usda.gov/rules-regulations/organic/national-list. Also found in this reference are materials not intended for inclusion in the compositions herein. The list is incorporated by reference herein in its entirety.

### Topical Compositions of the Disclosure

The present disclosure is directed to compositions for topical application, wherein the compositions comprise at least 95% organic ingredients. In some embodiments, the compositions comprise a cannabinoid, e.g., CBD in the form of a CBD oil or a CBD extract. In some embodiments, the compositions comprise the therapeutic oil blend of the disclosure, described in detail in the Therapeutic Oil Blends section herein. In embodiments, a composition comprises an oil-in-water emulsion composition comprised within a therapeutic oil blend. In some embodiments, the compositions comprise additional ingredients suited to their intended purpose and mode of administration. Such ingredients may include, e.g., essential oils, carrier oils, emulsifiers, humectants, flavoring, coloring, and the like. The compositions include, but are not limited to, lotions, body balms, massage oils, gel topicals, and salves.

### Topical Formulations

The compositions of the present disclosure are topical formulations. Non-limiting examples of the formulation include a lotion, a cream, a salve, a body balm, a liniment, an ointment, a gel, a paste, a tonic, an unguent, a nasal spray, a soap, a shampoo, and a lip balm.

In some embodiments, the composition is an oil-in-water emulsion. In some embodiments, the composition is a lotion. Unless otherwise indicated, the term “lotion” relates to a low- to medium-viscosity topical preparation intended for application to unbroken skin. In contrast, creams and gels have higher viscosity. Lotions are applied to external skin with bare hands, a clean cloth, cotton wool or gauze. Many lotions, especially hand lotions and body lotions are formulated not as a medicine delivery system, but simply to smooth, re-hydrate, and soften the skin. These are particularly popular with the aging and aged demographic groups, in the case of face usage, can also be classified as a cosmetic in many cases, and may contain fragrances.

In some embodiments, the compositions of the present disclosure have a viscosity of about 1000 cps, 1500 cps, 2000 cps, 2500 cps, 3000 cps, 3500 cps, 4000 cps, 4500 cps, 5000 cps, 5500 cps, 6000 cps, 6500 cps, 7000 cps, 7500 cps, or any ranges or subranges therebetween. In some embodiments, the composition of the present disclosure has a viscosity of about 4500-7000 cps. In some embodiments, the composition of the present disclosure has a viscosity of about 5000-6500 cps.

Most lotions are oil-in-water emulsions using emulsifiers to keep the emulsion together, but water-in-oil lotions are also formulated. The key components of a skin care lotion, cream or gel emulsion (that is mixtures of oil and water) are the aqueous and oil phases, an emulsifier to prevent separation of these two phases, and, if used, the drug substance or substances. Other ingredients are commonly added to lotions, such as fragrances, glycerol, petroleum jelly, dyes, preservatives, proteins and stabilizing agents.

In some embodiments, the composition of the present disclosure comprises a cannabinoid component. In some embodiments, the cannabinoid component is an oil, e.g., a CBD oil. In some embodiments, the cannabinoid component is provided by a therapeutic oil blend of the present disclosure. In some embodiments, the lotion comprises a therapeutic oil blend of the present disclosure comprising cannabidiol or a cannabidiol-related cannabinoid, a terpene, an emulsifier, a carrier oil, and an essential oil. In some embodiments, the composition does not comprise a therapeutic oil blend described herein and still forms a thermostable composition with a viscosity in the range of 400-7000 cps. In some embodiments, the composition comprises a therapeutic oil blend described herein and exhibits improved properties in terms of certain attributes, e.g., its therapeutic efficacy and absorption.

In some embodiments, the composition comprises rose oil. In some embodiments, the composition comprises menthol. In some embodiments, the composition comprises tea tree oil. In some embodiments, the composition comprises lavender oil. In some embodiments, the composition comprises camphor. In some embodiments, the composition comprises arnica oil. In some embodiments, the composition comprises black pepper oil. In some embodiments, the composition comprises turmeric oil. In some embodiments, the composition comprises peppermint oil. In some embodiments, the composition comprises sea buckthorn oil.

In some embodiments, the lotion comprises arnica oil. Arnica oil may be used for relieving pain associated with sore muscles, muscle aches, sprains, back and neck pain. Arnica oil may be beneficial for bruise treatment, insect bites, soothing sunburn, and to reduce inflammation in joints.

In some embodiments, the lotion comprises rose absolute oil. Rose absolute oil may be used to treat pain, anxiety, and depression. Rose absolute oil may have calming and/or sedative effects.

In some embodiments, the lotion comprises a carrier oil that is a plant-derived oil. In some embodiments, the lotion comprises one or more of the following plant-derived oils: almond oil, avocado seed oil, beech nut oil, bitter gourd oil, bottle gourd oil, brazil nut oil, buffalo gourd oil, butternut squash seed oil, canola oil, cashew oil, cocoa butter, coconut oil, corn oil, cottonseed oil, egusi seed oil, flax seed oil, grapefruit seed oil, grapeseed oil, hazelnut oil, hemp oil, lemon oil, macadamia oil, mongongo nut oil, olive oil, orange oil, palm oil, peanut oil, pecan oil, pine nut oil, pistachio oil, pumpkin seed oil, rapeseed oil, rice bran oil, safflower seed oil, sesame seed oil, sunflower seed oil, soybean oil, walnut oil, watermelon seed oil. In some embodiments, the lotion comprises olive oil, sunflower oil, and/or hempseed oil.

Lotions can be used for the delivery to the skin of medications such as: antibiotics; antiseptics; antifungals; corticosteroids; anti-acne agents; anti-inflammatory agents; anti-arthritis agents; anti-aging agents; soothing agents; anti-wrinkle agents; smoothing agents; moisturizing agents; and protective agents.

In some embodiments, the composition is a body butter, a body balm, or a salve. In general, body butters and body balms are made utilizing the base ingredients of natural butter(s) and herbal and/or vegetable/nut/seed oils. Balms typically contain beeswax. Salves typically comprise herbal oils and beeswax. Since body balms, butters, and salves do not contain water, they may be referred to as anhydrous formulations. The lack of water also means they do not require a preservative. In some embodiments, an antioxidant may be added, such as turmeric CO₂ oil, rosemary CO₂ extract, vitamin E or mixed tocopherols.

In some embodiments, a composition comprising the therapeutic oil blend of the present disclosure is a body balm. In some embodiments, the body balm may comprise one or more ingredients selected from the list consisting of: shea butter, beeswax, coconut oil, sunflower oil, jojoba oil, cocoa butter, MCT oil, turmeric oil, rosemary extract, vitamin E, and essential oils. In some embodiments, the body balm comprises turmeric oil. In some embodiments, the turmeric oil is CO₂ extracted turmeric oil. In some embodiments, the body balm has antioxidant effects. In some embodiments, the body balm has anti-inflammatory effects.

### Cannabinoids

The compositions of the present disclosure comprise one or more cannabinoids. The following discussion of cannabinoids is applicable equally to the compositions for topical application disclosed herein, e.g., lotions and balms, and the therapeutic oil blends disclosed herein, both of which are generically referred to as “composition” for the purposes of this section. Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C4 (THCA-C4), Tetrahydrocannbinol C4 (THC-C4), Tetrahydrocannabivarinic acid (THCVA), Tetrahydrocannabivarin (THCV), Tetrahydrocannabiorcolic acid (THCA-C4), Tetrahydrocannabiorcol (THC-C1), Δ7-cis-iso-tetrahydrocannabivarin, Δ8-tetrahydrocannabinolic acid (Δ8-THCA), Cannabivarinodiolic (CBNDVA), Cannabivarinodiol (CBNDV), Δ8-tetrahydrocannabinol (Δ8-THC), Δ9-tetrahydrocannabinol (Δ9-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodiolic Acid (CBNDA), Cannabinodivarin (CBDV), Cannabitriol (CBT), 10-Ethoxy-9-hydroxy-Δ6a-tetrahydrocannabinol, 8,9-Dihydroxy-Δ6a(10a)-tetrahydrocannabinol (8,9-Di-OH-CB T-C5), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide.

CBD is one of the active cannabinoids identified in cannabis. CBD does not appear to have any intoxicating effects such as those caused by THC in marijuana, but may have effects on anxiety, depression, psychological disorders, pain and post-traumatic stress disorder (PTSD), among other indications.

In some embodiments, the composition of the present disclosure comprises CBD. The terms “cannabidiol” and “CBD” are interchangeably used herein and refer to a non-psychotropic cannabinoid having structure as described in the formula below.

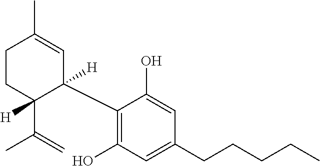 [SMILES: C=C(C)[C@@H]1CCC(C)=C[C@H]1c1c(O)cc(CCCCC)cc1O]

As used herein, the term “CBDA” refers to cannabidiolic acid and has the following structural formula:

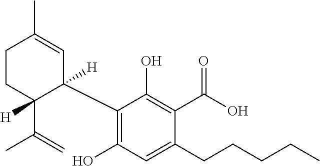 [SMILES: C=C(C)[C@@H]1CCC(C)=C[C@H]1c1c(O)cc(CCCCC)c(C(=O)O)c1O]

Decarboxylating CBDA with heat, light, etc., forms CBD and other possible cannabinoid derivatives.

As used herein, the term “CBDV” refers to cannabidivarin and has the following structural formula:

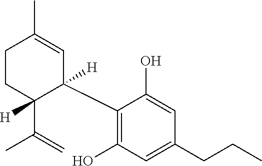 [SMILES: C=C(C)[C@@H]1CCC(C)=C[C@H]1c1c(O)cc(CCC)cc1O]

As used herein, the term “CBDVA” refers to cannabidivarinic acid and has the following structural formula:

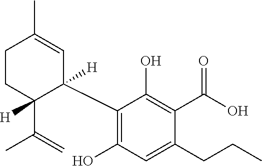 [SMILES: C=C(C)[C@@H]1CCC(C)=C[C@H]1c1c(O)cc(CCC)c(C(=O)O)c1O]

Decarboxylating CBDVA with heat, light, etc., forms CBDV and other possible cannabinoid derivatives.

In some embodiments, the composition comprises a cannabinoid selected from the group consisting of Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), salts thereof, derivatives thereof and mixtures thereof. Each possibility represents a separate embodiment of the disclosure. In some embodiments, the composition comprises Cannabidiolic Acid (CBDA). In some embodiments, the composition comprises Cannabidiol (CBD). In some embodiments, the composition comprises Cannabidiol monomethylether (CBDM). In some embodiments, the composition comprises Cannabidiol-C4 (CBD-C4). In some embodiments, the composition comprises Cannabidivarinic Acid (CBDVA). In some embodiments, the composition comprises Cannabidivarin (CBDV). In some embodiments, the composition comprises Cannabidiorcol (CBD-C1).

In some embodiments, the cannabinoid is a natural cannabinoid. In some embodiments, the cannabinoid is a natural cannabinoid found in a Cannabis plant. In some embodiments, the cannabinoid is a synthetic cannabinoid. In some embodiments, the cannabinoid is a mixture of natural cannabinoids. In some embodiments, the cannabinoid is a mixture of synthetic cannabinoids. In some embodiments, the cannabinoid is a mixture of natural and synthetic cannabinoids.

The term “natural cannabinoid” as used herein generally refers to a cannabinoid that can be found in, isolated from and/or extracted from a natural resource, such as plants. “Synthetic cannabinoids” are a class of chemicals that are different from the cannabinoids found e.g. in cannabis but which also bind to cannabinoid receptors.

Most cannabinoids exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). Cannabinoids in their acid forms (those ending in “-A”) can be converted to their non-acidic forms through a process called decarboxylation. While some decarboxylation (e.g., neutralization) of cannabinoids does occur in the plant, production of the neutral forms increase significantly post-harvest. (Sanchez and Verpoorte (2008) Plant Cell Physiol. December: 49(12)). Full decarboxylation of phytocannabinoids can be catalyzed by post-cultivation heating cannabis plant material or extracted cannabinoids (e.g., by combustion, vaporization, or baking in an oven).

Within the context of this application, where reference is made to a particular cannabinoid, each of the acid and/or decarboxylated forms are contemplated as both single molecules and mixtures.

In order to find the total amount of cannabinoids in a sample (e.g., total amount of active non-acidic cannabinoid), the total measured content of acid cannabinoid variants forms should be adjusted to account for the loss of the carboxyl group. In some embodiments, this adjustment can be made by multiplying the molar content of the acidic cannabinoid forms by the molecular weight of the corresponding decarboxylated cannabinoid. Other shorthand conversions are also available for quickly converting acidic cannabinoid content to active cannabinoid content.

For example, in some embodiments, THCA can be converted to active THC using the formula: THCA×0.877=THC. When using this approach, the maximum THC for the sample is: THCmax=(THCA×0.877)+THC. This method has been validated according to the principles of the International Conference on Harmonization. Similarly, CBDA can be converted to active CBD and the yield is determined using the yield formula: CBDA×0.877=CBD. Also, the maximum amount of CBD yielded, i.e. max CBD for the sample is: CBDmax=(CBDA×0.877)+CBD. Additionally, CBGA can be converted to active CBG by multiplying CBGA by 0.878 (CBGmax=(CBGA×0.878)+CBG).

Cannabinoids and terpenes from any source may be used in the compositions of the present disclosure. In some embodiments, cannabinoid and or terpenes are extracted from Cannabis plants. In some embodiments, the cannabinoids and terpenes described herein are provided as Cannabis extracts (also interchangeably referred to herein as “Cannabis plant extracts.”) In some embodiments, the cannabinoid and/or terpene extracts are produced from aerial parts of Cannabis plants, e.g., the stalks, stems, leaves, and seeds.

Cannabis plant extracts can be produced according to methods known in the art. For example, suitable extraction methods include maceration, percolation, solvent extraction, steam distillation (giving you essential oil) or vaporization. General protocols for the preparation of Cannabis extracts from cannabis plant material are described in U.S. Pat. Nos. 8,603,515 and 9,730,911, both incorporated by reference herein.

Solvent extraction may be carried out using essentially any solvent that dissolves cannabinoids/cannabinoid acids, such as for example C1 to C5 alcohols (e.g. ethanol, methanol), C4-C12 alkanes (e.g. hexane or butane), Norflurane (HFA134a), HFA227, and carbon dioxide. When solvents such as those listed above are used, the resultant primary extract typically contains non-specific lipid-soluble material or “ballast” e.g. waxes, wax esters and glycerides, unsaturated fatty acid residues, terpenes, carotenes, and flavonoids. The primary extract may be further purified for example by “winterization”, which involves chilling to −20° C. followed by filtration to remove waxy ballast, supercritical or subcritical extraction, vaporization, distillation, and chromatography.

Additional extraction techniques for cannabinoids, including vaporizer-based approaches, can be found in U.S. Pat. Nos. 7,700,368, 10,159,908, U.S. Pub. No. 2019/0151771, U.S. Pub. No. 2018/0078874, U.S. Pub. No. 2020/0080021, U.S. Pub. No. 2020/0048214, U.S. Pub. No. 2020/0048215, and U.S. patent Ser. No. 10/555,914, each of which is incorporated by reference herein in its entirety.

In some embodiments, terpenes are extracted from Cannabis using a vacuum-drying oven. Vacuum-drying ovens remove water, solvents, and terpenes from the Cannabis. The solution of water, solvents, and terpenes can be separated by filtration to purify terpenes.

In some embodiments, terpenes and/or cannabinoids are extracted from Cannabis using carbon dioxide. In some embodiments, the carbon dioxide is supercritical carbon dioxide (scCO₂). Carbon dioxide extraction may occur at very low temperatures, preventing compounds like terpenes and cannabinoids from degrading. U.S. Pat. No. 9,744,200 and International Application No. 2016/200438 describe carbon dioxide extraction processes and are each incorporated by reference herein in their entireties.

In some embodiments, the compositions of the present disclosure comprise one or more components are derived from sources other than the Cannabis plant (e.g., from other organisms, or chemically synthesized). For example, the compositions of the present disclosure can, in some embodiments, comprise cannabinoids and/or terpenes produced via standard chemical, biochemical, or biocatalytic methods. Persons having skill in the art will be familiar with various synthesis methods, including those of U.S. Pat. No. 9,359,625 and Taura et al. 1996, The Journal of Biological Chemistry, Vol. 271, No. 21, p. 17411-17416.

Additionally, cannabinoids and terpenes of the present disclosure can be commercially sourced. For example, CBD and THC can be purchased from Sigma-Aldrich Company Ltd, Fancy Road, Poole Dorset, BH12 4QH, or may be chemically synthesized. Beta-pinene and limonene can also be purchased from Sigma-Aldrich Company Ltd, Fancy Road, Poole Dorset, BH12 4QH.

Some cannabinoids do not accumulate at high levels in cannabis plant material. In some embodiments, these cannabinoids can be produced by chemical means. For example, in some embodiments, cannabinoids such as cannabinol are created from THC or CBD as described in Pollastro et al. and Adams et al. which are each incorporated by reference herein in their entirety: Pollastro et al. J. Nat. Prod. 2018, 81, 3, 630-633; Adams et al. J. Am. Chem. Soc. 1940, 62, 9, 2402-2405.

Cannabinoids used in compositions and methods of the present disclosure can be derived from various sources, including but not limited to hemp (e.g., hemp stalk, hemp stem,), cannabis (e.g., cannabis flower, cannabis leaf, cannabis stalk, cannabis stem,), Echinacea purpurea, Echinacea angustifolia, Echinacea pallida, Acmella oleracea, Helichrysum umbraculigerum, Radula marginata, kava, black truffle, Syzygium aromaticum (cloves), Rosmarinus oficinalis, basil, oregano, black pepper, lavender, true cinnamon, malabathrum, cananga odorata, Copaifera spp., and hops.

In some embodiments, the cannabinoids and terpenes of the present disclosure are pure isolates. In some embodiments, cannabinoids and/or terpenes are provided as a complex mixture (e.g., within a complex extract). A complex mixture contains two or more cannabinoids and/or terpenes. For example, in some embodiments, CBD and THC are provided as a solution containing 50% CBD and 50% THC.

In some embodiments, the composition is a topical formulation comprising the therapeutic oil blend of the disclosure. In some embodiments, the composition comprises additional cannabinoids. In some embodiments, the composition comprises additional terpenes.

In some embodiments, the compositions of the present disclosure comprise cannabinoids in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg), or more, per ounce (oz), including all ranges and subranges therebetween. In some embodiments, the compositions of the present disclosure comprise cannabinoids in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce (oz), including all ranges and subranges therebetween. In some embodiments, the compositions of the present disclosure comprise cannabinoids in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce (oz), including all ranges and subranges therebetween. In some embodiments, the compositions of the present disclosure comprise cannabinoids in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more by weight of the product, including all ranges and subranges therebetween. In some embodiments, the compositions of the present disclosure comprise cannabinoids in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product, including all ranges and subranges therebetween. In some embodiments, the compositions of the present disclosure comprise cannabinoids in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product, including all ranges and subranges therebetween.

In some embodiments, the composition for topical application of the present disclosure comprises cannabinoids in a weight percentage of about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1%, including all ranges and subranges therebetween. In some embodiments, the composition for topical application of the present disclosure comprises cannabinoids in a weight percentage of about 0.001%-0.01%. In some embodiments, the composition for topical application of the present disclosure is a lotion and comprises cannabinoids in a weight percentage of about 0.005%. In some embodiments, the composition for topical application of the present disclosure is a balm and comprises cannabinoids in a weight percentage of about 0.009%.

### Terpenes

The following discussion of terpenes is applicable to both the compositions for topical application disclosed herein, e.g., lotions and balms, and the therapeutic oil blends disclosed herein, both of which are generically referred to as “composition” for the purposes of this section. Terpenes are a large and diverse class of organic compounds, produced by a variety of plants, particularly conifers and citrus plants, and by some insects such as termites or swallowtail butterflies, which emit terpenes from their osmeteria. They often have a strong odor and may protect the plants that produce them by deterring herbivores and by attracting predators and parasites of herbivores.

When terpenes are modified chemically, such as by oxidation or rearrangement of the carbon skeleton, the resulting compounds are generally referred to as terpenoids. The difference between terpenes and terpenoids is that terpenes are hydrocarbons, whereas terpenoids contain additional functional groups. As used herein, the term “terpene” encompasses terpenoids. Terpenoids are also known as isoprenoids. Any terpene can be converted to a terpenoid, synthetic terpenoid or semisynthetic terpenoid by an array of known chemical reactions. These conversions have been taught exhaustively in the art.

Terpenes and terpenoids are the primary constituents of the essential oils of many types of plants and flowers. Essential oils are used widely as fragrances in perfumery, and in medicine and alternative medicines such as aromatherapy. Synthetic variations and derivatives of natural terpenes and terpenoids also greatly expand the variety of aromas used in perfumery and flavors used in food additives. Terpenes are a major constituent of Cannabis sativa plants, which contain at least 120 identified compounds.

Chemically, as used herein, the term “terpene” refers to a compound built on an isoprenoid structure or produced by combining isoprene units, 5 carbon structures. Within the context of this disclosure, the term “terpene” does not necessarily require 5 carbons or multiples of 5 carbons. It is understood that a reaction with isoprene units does not always result in a terpene comprising all the carbon atoms. Within the context of this disclosure, the term “terpene” includes cannabis-derived terpenes and non-cannabis-derived terpenes. Within the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. As well as isomeric, enantiomeric, or optically active derivatives. Within the context of this disclosure, the term terpene includes the α- (alpha), β- (beta), γ- (gamma), oxo-, isomers, or any combinations thereof. Terpenes can be acyclic, monocyclic, or polycyclic. Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids.

Examples of terpenes within the context of this disclosure include, without limitation: 7,8-dihydro-alpha-ionone, 7,8-dihydro-beta-ionone, acetanisole, acetic acid, acetyl cedrene, anethole, anisole, benzaldehyde, bergamotene (alpha-cis-bergamotene) (alpha-trans-bergamotene), bisabolol (beta-bisabolol), alpha bisabolol, borneol, bornyl acetate, butanoic/butyric acid, cadinene (alpha-cadinene) (gamma-cadinene), cafestol, caffeic acid, camphene, camphor, capsaicin, carene (delta-3-carene), carotene, carvacrol, dextro-carvone, laevo-carvone, alpha-caryophyllene, beta-caryophyllene, caryophyllene oxide, cedrene (alpha-cedrene) (beta-cedrene), cedrene epoxide (alpha-cedrene epoxide), cedrol, cembrene, chlorogenic acid, cinnamaldehyde, alpha-amyl-cinnamaldehyde, alpha-hexyl-cinnamaldehyde, cinnamic acid, cinnamyl alcohol, citronellal, citronellol, cryptone, curcumene (alpha-curcumene) (gamma-curcumene), decanal, dehydrovomifoliol, diallyl disulfide, dihydroactinidiolide, dimethyl disulfide, eicosane/lcosane, elemene (beta-elemene), estragole, ethyl acetate, ethyl cinnamate, ethyl maltol, eucalyptol/1,8-cineole, eudesmol (alpha-eudesmol) (beta-eudesmol) (gamma-eudesmol), eugenol, euphol, farnesene, farnesol, fenchol (beta-fenchol), fenchone, geraniol, geranyl acetate, germacrenes, germacrene b, guaia-1(10),11-diene, guaiacol, guaiene (alpha-guaiene), gurjunene (alpha-gurjunene), herniarin, hexanaldehyde, hexanoic acid, hexyl acetate humulene (alpha-humulene) (beta-humulene), ionol (3-oxo-alpha-ionol) (beta-ionol), ionone (alpha-ionone) (beta-ionone), ipsdienol, isoamyl acetate, isoamyl alcohol, isoamyl formate, isoborneol, isomyrcenol, isopulegol, isovaleric acid, isoprene, kahweol, lavandulol, limonene, gamma-linolenic acid, linalool, longifolene, alpha-longipinene, lycopene, menthol, methyl butyrate, 3-mercapto-2-methylpentanal, mercaptan/thiols, beta-mercaptoethanol, mercaptoacetic acid, allyl mercaptan, benzyl mercaptan, butyl mercaptan, ethyl mercaptan, methyl mercaptan, furfuryl mercaptan, ethylene mercaptan, propyl mercaptan, thenyl mercaptan, methyl salicylate, methylbutenol, methyl-2-methylvalerate, methyl thiobutyrate, myrcene (beta-myrcene), gamma-muurolene, nepetalactone, nerol, nerolidol, neryl acetate, nonanaldehyde, nonanoic acid, ocimene, octanal, octanoic acid, p-cymene, pentyl butyrate, phellandrene, phenylacetaldehyde, phenylethanethiol, phenylacetic acid, pinene, alpha-pinene, beta-pinene, propanethiol, pristimerin, pulegone, phytolquercetin, retinol, rutin, sabinene, sabinene hydrate, cis-sabinene hydrate, trans-sabinene hydrate, safranal, alpha-selinene, alpha-sinensal, beta-sinensal, beta-sitosterol, squalene, taxadiene, terpin hydrate, terpineol, terpine-4-ol, alpha-terpinene, gamma-terpinene, terpinolene, thiophenol, thujone, thymol, alpha-tocopherol, tonka undecanone, undecanal, valeraldehyde/pentanal, verdoxan, alpha-ylangene, umbelliferone, or vanillin. Additional examples of terpenes and their characteristics may be found in U.S. Publication No. 20170266153, herein incorporated by reference in its entirety.

Terpenes in compositions of the present disclosure can be selected to provide benefits for particular conditions or subjects. In some embodiments, the terpene or the mixture of terpenes solubilize a cannabinoid or a mixture of cannabinoids. Each possibility represents a separate embodiment of the disclosure. In some embodiments, terpenes can be employed in combination with each other, as well as in combination with cannabinoids, to ameliorate one or more health conditions. For example, terpinolene, terpineol and linalool or lavender, valerian and jasmine essential oils can be combined with cannabinoids or cannabis extract to act as a sleep aid or treat sleep disorders. Beneficially, the combination of cannabinoids and terpenes, such as in a single composition, can have a synergistic effect on a subject's endocannabinoid system. For example, the presence of the terpenes can increase bioavailability of the cannabinoids. Alternatively or in addition, the presence of the cannabinoids can increase bioavailability of the terpenes.

In some embodiments, the composition comprises about 0.05% to about 80% by weight of a terpene or a mixture thereof. In some embodiments, the composition comprises about 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, or 60% by weight of a terpene or a mixture thereof, including all ranges and subranges therebetween. In some embodiments, the composition comprises about 0.1% to about 50% by weight of a terpene or a mixture thereof. In some embodiments, the composition comprises about 5% to about 20% by weight of a terpene or a mixture of terpenes. In some embodiments, the composition comprises about 8-10% by weight of a terpene or mixture of terpenes.

### Essential Oils

The compositions of the present disclosure can comprise one or more essential oils or essential oil compounds. Essential oils can include, but are not limited to: Linalool; B-Caryophyllene; B-Myrcene; D-Limonene; Humulene; a-Pinene; Ylang Ylang (Cananga odorata); Yarrow (Achillea millefolium); Violet (Viola odorata); Vetiver (Vetiveria zizanoides); Vanilla (Vanilla plantifolia); Tuberose (Polianthes tuberosa); Thyme (Thymus vulgaris L.); Tea Tree (Melaleuca alternifolia); Tangerine (Citrus reticulata); Spruce, Black (Picea mariana); Spruce (Tsuga Canadensis); Spikenard (Nardostachys jatamansi); Spearmint (Mentha spicata); Sandalwood (Santalum spicatum); Rosewood (Aniba rosaeodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestris L.); Petitgrain (Citrus aurantium); Peppermint (Mentha piperita); Pepper, Black (Piper nigrum L.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evernia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L.); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citrus aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus communis); Jasmine (Jasminum officinale); Jasmine Abs (Jasminum sambac); Helichrysum (Helichrysum italicum); Grapefruit, White (Citrus×paradisi); Grapefruit, Pink (Citrus paradisi); Ginger (Zingiber officinalis); Geranium (Pelargonium graveolens); Geranium, Bourbon (Pelargonium graveolens, 'Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies siberica); Fir Needle Canada (Abies balsamea); Fennel, Sweet (Foeniculum vulgare); Eucalyptus Smithii. Eucalyptus Radiata, Eucalyptus Globulus, Eucalyptus Citriodora, Eucalyptus Blue Mallee (Eucalyptus polybractea); Elemi (Canarium luzonicum); Dill (Anethum graveolens); Cypress (Cupressus sempervirens); Cumin (Cuminum cyminum); Coriander (Coriandum sativum); Cocoa (Theobroma cacao); Clove (Eugenia caryophylatta); Clary Sage (Salvia sclarea); Cistus (aka Labdanum) (Cistus ladaniferus L.); Cinnamon (Cinnamomum zeylanicum); Chamomile, Roman (Anthemis nobilis); Chamomile, Blue (Matricaria chamomilla); Celery Seed (Apium graveolins); Cedarwood, Western Red (Thuja plicata); Cedarwood, Blood (Juniperus virginiana); Cedarwood Atlas (Cedrus atlantica); Carrot Seed (Daucus carota); Cardamon (Elettaria cardamomum); Caraway Seed (Carum carvi); Cajeput (Melaleuca cajuputi); Cade (Juniperus oxycedrus); Birch, White (Betula alba); Birch, Sweet (Betula lenta); Bergamot (Citrus bergamia); Bay Laurel (Laurus nobilis); Basil (Ocimum basilicum); Basil, Holy (Ocimum sanctum); Basil (Ocimum basilicum); Balsam Poplar (Populus balsamifera); Balsam Peru (Myroxylon balsamum); Angelica (Angelica archangelica L.); and/or combinations thereof. In embodiments, the essential oil is selected from the group consisting of: rose oil, menthol, tea tree oil, lavender, camphor, arnica, black pepper oil, turmeric, peppermint, eucalyptus oil, lemon balm, chamomile oil, clove oil, rose geranium oil, sea buckthorn oil, and combinations thereof.

In some embodiments, the composition comprising the therapeutic oil blend of the present disclosure may comprise an essential oil derived from a plant selected from the following list: Alfalfa (Medicago sativa L.); Allspice (Pimenta officinalis Lindl.); Almont, bitter (pure from prussic acid) (Prunnus amygdalus Batsch, Prussun armeniaca L., or Prunnus persica (L.) Batsch.); Ambrette (seed) (Hibiscus moschatus Moench.); Angelica root (Angelica archangelica L.); Angelica seed; Angelica stem; Angostura (cusparia bark) (Galipea officinalis Hancock.); Anise (Pimpinella anisum L.); Asafetida (Ferula assa-foetida L. and related spp. of Ferula.); Balm (lemon balm) (Melissa officinalis L.); Balsam of Peru (Myroxylon pereirae Klotzsch.); Basil (Ocimum basilicum L.); Bay leaves (Laurus nobilis L.); Bay (myrcia oil) (Pimenta racemosa (Mill.) J. W. Moore.); Bergamot (bergamot orange) (Citrus aurantium L. subsp. bergamia Wright et Am.); Bitter almond (free from prussic acid) (Prunus amygdalus Batsch, Prunus armeniaca L., or Prunus persica (L.) Batsch.); Bois de rose (Aniba rosaeodora Ducke.); Cacao (Theobroma cacao L.); Camomile (chamomile) flowers, Hungarian (Matricaria chamomilla L.); Camomile (chamomile) flowers, Roman or English (Anthemis nobilis L.); Cananga (Cananga odorata Hook. f and Thoms.); Capsicum (Capsicum frutescens L. and Capsicum annuum L.); Caraway (Carum carvi L.); Cardamom seed (cardamon) (Elettaria cardamomum Maton.); Carob bean (Ceratonia siliqua L.); Carrot (Daucus carota L.); Cascarilla bark (Croton eluteria Benn.); Cassia bark, Chinese (Cinnamomum cassia Blume.); Cassia bark, Padang or Batavia (Cinnamomum burmanni Blume.); Cassia bark, Saigon (Cinnamomum loureirii Nees.); Celery seed (Apium graveolens L.); Cherry, wild, bark (Prunus serotina Ehrh.); Chervil (Anthriscus cerefolium (L.) Hoffm.); Chicory (Cichorium intybus L.); Cinnamon bark, Ceylon (Cinnamomum zeylanicum Nees.); Cinnamon bark, Chinese (Cinnamomum cassia Blume.); Cinnamon bark, Saigon (Cinnamomum loureirii Nees.); Cinnamon leaf, Ceylon (Cinnamomum zeylanicum Nees.); Cinnamon leaf, Chinese (Cinnamomum cassia Blume.); Cinnamon leaf, Saigon (Cinnamomum loureirii Nees.); Citronella (Cymbopogon nardus Rendle.); Citrus peels (Citrus spp.); Clary (clary sage) (Salvia sclarea L.); Clover (Trifolium spp.); Coca (decocainized) (Erythroxylum coca Lam. and other spp. of Erythroxylum.); Coffee (Coffea spp.); Cola nut (Cola acuminata Schott and Endl., and other spp. of Cola.); Coriander (Coriandrum sativum L.); Cumin (cummin) (Cuminum cyminum L.); Curacao orange peel (orange, bitter peel) (Citrus aurantium L.); Cusparia bark (Galipea officinalis Hancock.); Dandelion (Taraxacum officinale Weber and T. laevigatum DC.); Dandelion root; Dog grass (quackgrass, triticum) (Agropyron repens (L.) Beauv.); Elder flowers (Sambucus canadensis L. and S. nigra I.); Estragole (esdragol, esdragon, tarragon) (Artemisia dracunculus L.); Estragon (tarragon); Fennel, sweet (Foeniculum vulgare Mill.); Fenugreek (Trigonella foenum-graecum L.); Galanga (galangal) (Alpinia officinarum Hance.); Geranium (Pelargonium spp.); Geranium, East Indian (Cymbopogon martini Stapf.); Geranium, rose (Pelargonium graveolens L′Her.); Ginger (Zingiber officinale Rosc.); Grapefruit (Citrus paradisi Macf.); Guava (Psidium spp.); Hickory bark (Carya spp.); Horehound (hoarhound) (Marrubium vulgare L.); Hops (Humulus lupulus L.); Horsemint (Monarda punctata L.); Hyssop (Hyssopus officinalis L.); Immortelle (Helichrysum augustifolium DC.); Jasmine (Jasminum officinale L. and other spp. of Jasminum.); Juniper (berries) (Juniperus communis L.); Kola nut (Cola acuminata Schott and Endl., and other spp. of Cola.); Laurel berries (Laurus nobilis L.); Laurel leaves (Laurus spp.); Lavender (Lavandula officinalis Chaix.); Lavender, spike (Lavandula latifolia Vill.); Lavandin (Hybrids between Lavandula officinalis Chaix and Lavandula latifolin Vill.); Lemon (Citrus limon (L.) Burm. f.); Lemon balm (see balm); Lemon grass (Cymbopogon citratus DC. and Cymbopogon lexuosus Stapf.); Lemon peel (Citrus limon (L.) Burm. f.); Lime (Citrus aurantifolia Swingle.); Linden flowers (Tilia spp.); Locust bean (Ceratonia siliqua L,); Lupulin (Humulus lupulus L.); Mace (Myristica fragrans Houtt.); Mandarin (Citrus reticulata Blanco.); Marjoram, sweet (Majorana hortensis Moench.); Mate (Ilex paraguariensis St. Hil.); Melissa (see balm); Menthol (Mentha spp.); Menthyl acetate; Molasses (extract) (Saccarum officinarum L.); Mustard (Brassica spp.); Naringin (Citrus paradisi Macf.); Neroli, bigarade (Citrus aurantium L.); Nutmeg (Myristica fragrans Houtt.); Onion (Allium cepa L.); Orange, bitter, flowers (Citrus aurantium L.); Orange, bitter, peel; Orange leaf (Citrus sinensis (L.) Osbeck.); Orange, sweet; Orange, sweet, flowers; Orange, sweet, peel; Origanum (Origanum spp.); Palmarosa (Cymbopogon martini Stapf.); Paprika (Capsicum annuum L.); Parsley (Petroselinum crispum (Mill.) Mansf); Pepper, black (Piper nigrum L.); Pepper, white; Peppermint (Mentha piperita L.); Peruvian balsam (Myroxylon pereirae Klotzsch.); Petitgrain (Citrus aurantium L.); Petitgrain lemon (Citrus limon (L.) Burm. f.); Petitgrain mandarin or tangerine (Citrus reticulata Blanco.); Pimenta (Pimenta officinalis Lindl.); Pimenta leaf (Pimenta officinalis Lindl.); Pipsissewa leaves (Chimaphila umbellata Nutt.); Pomegranate (Punica granatum L.); Prickly ash bark (Xanthoxylum (or Zanthoxylum) Americanum Mill. or Xanthoxylum clava-herculis L.); Rose absolute (Rosa alba L., Rosa centifolia L., Rosa damascena Mill., Rosa gallica L., and vars. of these spp.); Rose (otto of roses, attar of roses); Rose buds; Rose flowers; Rose fruit (hips); Rose geranium (Pelargonium graveolens L'Her.); Rose leaves (Rosa spp.); Rosemary (Rosmarinus officinalis L.); Saffron (Crocus sativus L.); Sage (Salvia officinalis L.); Sage, Greek (Salvia triloba L.); Sage, Spanish (Salvia lavandulaefolia Vahl.); St. John's bread (Ceratonia siliqua L.); Savory, summer (Satureia hortensis L.); Savory, winter (Satureia montana L.); Schinus molle (Schinus molle L.); Sloe berries (blackthorn berries) (Prunus spinosa L.); Spearmint (Mentha spicata L.); Spike lavender (Lavandula latifolia Vill.); Tamarind (Tamarindus indica L.); Tangerine (Citrus reticulata Blanco.); Tarragon (Artemisia dracunculus L.); Tea (Thea sinensis L.); Thyme (Thymus vulgaris L. and Thymus zygis var. gracilis Boiss.); Thyme, white; Thyme, wild or creeping (Thymus serpyllum L.); Triticum (see dog grass); Tuberose (Polianthes tuberosa L.); Turmeric (Curcuma longa L.); Vanilla (Vanilla planifolia Andr. or Vanilla tahitensis J. W. Moore.); Violet flowers (Viola odorata L.); Violet leaves; Violet leaves absolute; Wild cherry bark (Prunus serotina Ehrh.); Ylang-ylang (Cananga odorata Hook. f. and Thorns.); and Zedoary bark (Curcuma zedoaria Rose.).

In some embodiments, the composition comprises about 0.1%-10% by weight of each essential oil. For example, in some embodiments, the composition comprises about 0.3%-2.0% by weight of an essential oil. In some embodiments, the composition comprises about 0.3% by weight of an essential oil. In some embodiments, the composition comprises about 1.0% by weight of an essential oil. In some embodiments, the composition comprises about 1.5% by weight of an essential oil. In some embodiments, the composition comprises about 3.0% by weight of an essential oil.

In some embodiments, the composition comprises rose oil. In some embodiments, the composition comprises menthol. In some embodiments, the composition comprises tea tree oil. In some embodiments, the composition comprises lavender oil. In some embodiments, the composition comprises camphor. In some embodiments, the composition comprises arnica oil. In some embodiments, the composition comprises black pepper oil. In some embodiments, the composition comprises turmeric oil. In some embodiments, the composition comprises peppermint oil. In some embodiments, the composition comprises sea buckthorn oil.

In some embodiments, the composition comprises one or more alcohol-based plant extracts. In some embodiments, the composition comprises one or more alcohol-based plant extracts in a weight percent of about 0.1-10.0%, about 0.3-3.0%, about 0.3%, or about 3.0%. In some embodiments, the composition comprises lemon balm extract. In some embodiments, the composition comprises chamomile extract.

### Carrier Oils

In some embodiments, a composition according to the present disclosure comprises a carrier oil. In some embodiments, the carrier is a vegetable oil. In some embodiments, the carrier oil is an essential oil. In some embodiments, the carrier oil is organic. Various exemplary carrier oils and essential oils are described, for example, in U.S. Pat. No. 10,806,707, the disclosure of which is incorporated by reference herein in its entirety, and any of these oils may be used in the compositions of the present disclosure.

In some embodiments, the carrier oil is medium chain triglyceride (MCT) oil, long chain triglyceride (LCT) oil, coconut oil, corn oil, canola oil, olive oil, avocado oil, vegetable oil, flaxseed oil, palm oil, peppermint oil, hemp oil, sesame oil, sunflower oil, a winterized oil of long-chain mono-, di-, and tri-glycerides (e.g. Maisine® CC), rice bran oil, or any combination thereof. In some embodiments, the carrier oil is almond oil; aloe vera oil; apricot kernel oil; avocado oil; argan oil; calendula oil; carrot seed oil; castor oil; coconut oil; evening primrose oil; fish oils and oils rich in omega-3 fatty acids (e.g., algae, krill, flaxseed); grape seed oil; hazelnut oil; hemp seed oil; jojoba oil; macadamia oil; olive oil; raspberry seed oil; sesame oil; sunflower oil; walnut oil; wheatgerm oil, or any combination thereof. In embodiments, the carrier oil comprises an oil selected from the group consisting of: sunflower oil, hemp oil or hemp seed oil, olive oil, and combinations thereof.

In some embodiments, a carrier oil is present in the composition in an amount ranging from about 0.5% to about 99.5%. In some embodiments, a carrier oil may be present in the therapeutic oil blend composition in an amount of about 0.1% (w/w), 0.5 (w/w), 1% (w/w), 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w), about 80% (w/w), about 85% (w/w), about 90% (w/w), about 95% (w/w), about 96% (w/w), about 97% (w/w), about 98% (w/w), including all ranges and subranges therebetween.

In embodiments, a carrier oil is present in the composition in an amount ranging from about 0.1% to 40% by weight. In embodiments, a carrier oil is present in the composition in an amount ranging from about 1% to 30% by weight. In embodiments, the carrier oil is present in the composition in an amount ranging from about 5% to 20% by weight. In embodiments, the carrier oil is present in the composition in an amount ranging from about 10% to 20% by weight. In embodiments, the carrier oil is present in the composition in an amount ranging from about 11% to 30% by weight. In embodiments, the carrier oil in the composition is present in an amount selected from the group consisting of: 1-10% sunflower oil, 0.1-2% hemp oil or hemp seed oil, 1-10% olive oil, and combinations thereof.

In some embodiments, the composition comprises olive oil. In some embodiments, the composition comprises 1-15% w/w olive oil. In some embodiments, the composition comprises 2-8% w/w olive oil. In some embodiments, the composition comprises about 5.6% w/w olive oil.

In some embodiments, the composition comprises sunflower oil. In some embodiments, the composition comprises 1-15% w/w sunflower oil. In some embodiments, the composition comprises 2-8% w/w sunflower oil. In some embodiments, the composition comprises about 5.6% w/w sunflower oil.

In some embodiments, the composition comprises hemp seed oil. In some embodiments, the composition comprises 0.1-15% w/w hemp seed oil. In some embodiments, the composition comprises 0.1-8% w/w hemp seed oil. In some embodiments, the composition comprises about 0.6% or about 5.6% w/w hemp seed oil.

In some embodiments, the composition comprises two or more of olive oil, sunflower oil, and hemp seed oil. In some embodiments, the composition comprises each of olive oil, sunflower oil, and hemp seed oil.

In some embodiments, the composition comprises MCT oil. In some embodiments, the composition comprises 20-35% w/w MCT oil. In some embodiments, the composition comprises 25-29% MCT oil. In some embodiments, the composition comprises about 27% MCT oil.

### Emulsifiers

In some embodiments, the composition comprises an emulsifier (e.g. emulsion stabilizer). In some embodiments, the emulsifier is organic. In some embodiments, the composition comprises a mixture of emulsifiers. In some embodiments, emulsifiers are foaming agents. In some embodiments, the emulsifier is saponin, sodium alginate, or guar gum. In some embodiments, the composition comprises saponin, sodium alginate, and guar gum. In some embodiments, the composition comprises each of saponin, sodium alginate, and guar gum. In some embodiments, saponin acts as a surfactant and foaming agent within the composition. In some embodiments, the foaming agent properties of saponin are used to aerate and lighten the consistency of the composition. In some embodiments, sodium alginate and/or guar gum act as thickening agents and emulsifiers within the composition. In embodiments, the emulsion stabilizer comprises sodium alginate or sodium alginate and Guar Gum.

In some embodiments, the composition comprises about 0.5% to about 5% w/w of an emulsifier or a mixture of emulsifiers. In some embodiments, the composition comprises about 0.1-1.0% w/w guar gum. In some embodiments, the composition comprises about 0.3% w/w guar gum. In some embodiments, the composition comprises about 0.1-5.0% w/w sodium alginate. In some embodiments, the composition comprises about 1.0% w/w sodium alginate. In some embodiments, the composition comprises about 0.1-5.0% w/w saponin. In some embodiments, the composition comprises about 1.0% w/w saponin. In embodiments, the emulsion stabilizer comprises 0.1-5.0% sodium alginate by weight, or 0.1-5% sodium alginate by weight and 0.1-1% Guar Gum by weight. In embodiments, the emulsion stabilizer comprises 1.0% sodium alginate by weight, or 1% sodium alginate by weight and 0.3% Guar Gum by weight.

Other examples of emulsifiers suitable for use in some embodiments of the disclosure are selected from the group consisting of polysorbate 80, oleoyl polyoxyl-6 glycerides, polyoxyl 35 hydrogenated castor oil, sucrose distearate, tocopherol polyethylene glycol 1000 succinate, lauroyl polyoxyl-32 glycerides, sorbitan monooleate, glyceryl stearate, cetearyl alcohol, sodium stearoyl lactylate, salts thereof, derivatives thereof, and mixtures of emulsifiers.

In some embodiments, emulsifier components are selected from the group consisting of poly-glycolized glycerides and polyoxyethylene glycerides of medium to long chain mono-, di-, and triglycerides, such as: almond oil PEG-6 esters, almond oil PEG-60 esters, apricot kernel oil PEG-6 esters (Labrafil® M1944CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-50 esters, hydrogenated castor oil PEG-5 esters, hydrogenated castor oil PEG-7 esters, 9 hydrogenated castor oil PEG-9 esters, corn oil PEG-6 esters (Labrafil® M 2125 CS), corn oil PEG-8 esters (Labrafil® WL 2609 BS), corn glycerides PEG-60 esters, olive oil PEG-6 esters (Labrafil® M1980 CS), hydrogenated palm/palm kernel oil PEG-6 esters (Labrafil® M 2130 BS), hydrogenated palm/palm kernel oil PEG-6 esters with palm kernel oil, PEG-6, palm oil (Labrafil® M 2130 CS), palm kernel oil PEG-40 esters, peanut oil PEG-6 esters (Labrafil® M 1969 CS), glycerol esters of saturated C8-C18 fatty acids (Gelucire® 33/01), glyceryl esters of saturated C12-C18 fatty acids (Gelucire® 39/01 and 43/01), glyceryl laurate/PEG-32 laurate (Gelucire® 44/14), glyceryl laurate glyceryl/PEG 20 laurate, glyceryl laurate glyceryl/PEG 32 laurate, glyceryl, laurate glyceryl/PEG 40 laurate, glyceryl oleate/PEG-20 glyceryl, glyceryl oleate/PEG-30 oleate, glyceryl palmitostearate/PEG-32 palmitostearate (Gelucire® 50/13), glyceryl stearate/PEG stearate, glyceryl stearate/PEG-32 stearate (Gelucire® 53/10), saturated polyglycolized glycerides (Gelucire® 37/02 and Gelucire® 50/02), triisostearin PEG-6 esters (i.e. Labrafil® Isostearique), triolein PEG-6 esters, trioleate PEG-25 esters, polyoxyl 35 castor oil (Cremophor® EL or Kolliphor® EL), polyoxyl 40 hydrogenated castor oil (Cremophor® RH 40 or Kolliphor® RH40), polyoxyl 60 hydrogenated castor oil (Cremophor® RH60), lecithin, phospholipids and mixtures thereof.

In some embodiments, the emulsifier is polyglycolized derivatives and polyoxyethylene esters or ethers derivatives of medium to long chain fatty acids, commercially named Brij and Myrj variety surfactants, and propylene glycol esters of medium to long chain fatty acids, which can be used including caprylate/caprate diglycerides, glyceryl monooleate, glyceryl ricinoleate, glyceryl laurate, glyceryl dilaurate, glyceryl dioleate, glyceryl mono/dioleate, glyceryl caprylate/caprate, medium chain (C8/C10) mono- and diglycerides (Capmul® MCM, Capmul® MCM (L)), mono- and diacetylated monoglycerides, polyglyceryl oleate, polyglyceryl-2 dioleate, polyglyceryl-10 trioleate, polyglyceryl-10 laurate, polyglyceryl-10 oleate, and polyglyceryl-10 mono dioleate, propylene glycol caprylate/caprate (Labrafac® PC), propylene glycol dicaprylate/dicaprate (Miglyol® 840), propylene glycol monolaurate, propylene glycol ricinoleate, propylene glycol monooleate, propylene glycol dicaprylate/dicaprate, propylene glycol dioctanoate, and mixtures thereof.

### Humectants

In some embodiments, the composition comprises a humectant. In some embodiments, the humectant is organic. In some embodiments, the composition is a topical formulation and comprises a humectant, which can be referred to as a soothing, smoothing, moisturizing or protective agent. Humectants of the present disclosure function to stabilize the moisture content of the tissue to which it is applied in the presence of fluctuating humidity.

In some embodiments, the humectant is glycerin. In some embodiments, the glycerin is present in the composition in a range of 1.0%-10% w/w. In some embodiments, the glycerin is present in the composition in a weight percent of about 1.0%, 2.0%, 3.0%, 4.0%, 5.0%, 6.0%, 7.0%, 8.0%, 9.0%, 10.0%, including all ranges and subranges therebetween. In some embodiments, the glycerin is present in the composition in a weight percent of about 5%.

Other examples of suitable humectants for use in the compositions of the present disclosure according to some embodiments include: polyglycols (as hereinafter defined), propylene glycol, sorbitol, lactic acid, sodium lactate, glycerol, ethoxylated castor oil, calamine, dodecylsulphate, sodium lauryl sulphate (SLS); a polyoxyethylene ester of polysorbitan, such as monooleate, monolaurate, monopalmitate, monostearate esters; esters of sorbitan, the polyoxyethylenes ethers, sodium dioctylsulphosuccinate (DOSS), lecithin, sodium docusate, hexylene glycol, butylene glycol, aloe vera gel, aloe vera powder, hyaluronic acid, alpha hydroxy acids such as lactic acid, egg yolk, egg white, glyceryl triacetate, honey, molasses, polymeric polyols such as polydextrose, quillaia, sodium hexametaphosphate e452i; sugar alcohols (sugar polyols) such as glycerol, sorbitol, xylitol, maltitol; urea, and castor oil.

The amount of the humectant in a topical formulation is not particularly limited, so long as it is a therapeutically effective amount. In some embodiments, the humectant is present in a composition in a range of 0.01-10% w/w. In some embodiments, the humectant, e.g., glycerin, is present in a composition, e.g., lotion, at about 5% w/w.

### Foaming Agent

In embodiments, a composition provided herein comprises a foaming agent. In embodiments, the composition comprises an oil-in-water emulsion composition that comprises a foaming agent. Exemplary foaming agents include but are not limited to: saponin, azodicarbonamide, barium azodicarboxylate, azobisisobutyronitrile, azodicarboxylic acid amide, N, N′-dinitrosopentamethylenetetramine, N, N′-dimethyl-N, N′—. Dinitrosoterephthalamide, trinitrotrimethyltriamine, 4,4′-oxybis (benzenesulfonylhydrazide), paratoluenesulfonylhydrazide, diphenylsulfone-3,3′-disulfonylhydrazide, allylbis (sulfonylhydrazide), p-toluylenesulfonyl Fluorinated alkanes such as semicarbazide, 4,4′-oxybis (benzenesulfonyl semicarbazide), trichloromonofluoromethane, dichloromonofluoromethane, 5-morpholyl-1,2,3,4-thiatriazole, carbonate Bromide, ammonium bicarbonate, sodium bicarbonate, ammonium nitrite, sodium borohydride, azides, modified versions thereof, and combinations thereof.

In embodiments, the foaming agent comprises saponin. In embodiments, saponin is present in an amount from about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, up to about 5% by weight. In embodiments, the foaming agent comprises saponin at 1% by weight.

### Additional Ingredients

The compositions of the present disclosure can comprise an additional agent or agents, whether active or passive. Examples of such an agent include a sweetening agent, a flavoring agent, a coloring agent, a filling agent, a binding agent, a lubricating agent, an excipient, a preservative, an emollient, a hydrating agent, a smoothing agent, or a manufacturing agent. Additional pharmaceutically acceptable excipients (in the case of pharmaceuticals) or other additives (for non-pharmaceutical applications) can be added to the composition. For example, if desired, any generally accepted soluble or insoluble inert pharmaceutical filler (diluent) material can be included in the final product (e.g., a solid dosage form). Such inert pharmaceutical filler can comprise a monosaccharide, a disaccharide, a polyhydric alcohol, inorganic phosphates, sulfates or carbonates, and combinations thereof. Examples of suitable inert pharmaceutical fillers include sucrose, dextrose, lactose, xylitol, fructose, sorbitol, calcium phosphate, calcium sulfate, calcium carbonate, microcrystalline cellulose, and combinations thereof. An effective amount of any generally accepted pharmaceutical lubricant, such as calcium or magnesium soaps, can be added.

Depending on the dosage form, optional additives and modifiers further comprise one or more of acids, bases, acidity regulators, alcohol, anticaking agents, antifoaming agents, antioxidants, bulking agents, coagulation agents, colour retention agents, emulsifiers, flavor enhancers, flour treatment agents, gelling agents, glazing agents, humectants, leavening agents, tracer gases, preservatives, stabilizers, sweeteners, tenderizers, and thickeners.

Additional herbal extracts, essential oils, homeopathic remedies, and flower essences are known and may also be added to the therapeutic oil blend compositions of the present disclosure. Examples of such are provided in U.S. Publication No. 20180344661, the disclosure of which is incorporated herein by reference in its entirety. The therapeutic oil blend composition may also comprise a fruit extract, such as an extract of coconuts, apricots, apples, pears, peaches, pineapples, papayas, pomegranates, cherries, kiwis, tangerines, oranges, grapes, or mixtures thereof. Additional fruit extracts and additives may be found in, e.g., U.S. Pat. No. 6,630,163, the disclosure of which is incorporated by reference herein in its entirety.

Other ingredients may also be present in the composition, such as antibiotics; antiseptics; antifungals; corticosteroids; soothing agents; anti-aging agents; smoothing agents; moisturizing agents; and protective agents.

### Effective Ratios of Different Phases of Compositions

Lotions are broadly composed of three ingredient phases: a water phase, an oil phase, and a preservative/alcohol phase.

In some embodiments, a lotion of the disclosure comprises about 40-60% ingredients in the water phase. In some embodiments, a lotion of the disclosure comprises about 45-57% ingredients in the water phase. In some embodiments, a lotion of the disclosure comprises about 15-30% ingredients in the oil phase. In some embodiments, a lotion of the disclosure comprises about 20-28% ingredients in the oil phase. In some embodiments, a lotion of the disclosure comprises about 15-30% ingredients that are alcohols or alcohol extracts. In some embodiments, a lotion of the disclosure comprises about 18-26% ingredients that are alcohols or alcohol extracts. In some embodiments, a lotion of the disclosure comprises about 1-5% solids such as sodium alginate, saponin, and guar gum.

The present application is based, in part, on the unexpected discovery of an effective ratio of ingredients in the water phase, oil phase, and preservative/alcohol phase of the cannabinoid-containing lotions of the disclosure that was able to maintain thermostability and consistency. In some embodiments, it was determined through extensive experimentation that a percentage by mass of about 22% oil phase, about 55% water phase, and about 20% alcohol phase was effective in encouraging desirable properties of the lotion. In some embodiments, the remaining 2.3% of the lotion was composed of sodium alginate, saponin, and guar gum. In some embodiments, it was determined through extensive experimentation that a percentage by mass of about 26% oil phase, about 47% water phase, and about 24% alcohol phase was effective in encouraging desirable properties of the lotion. In some embodiments, the remaining 2.3% of the lotion was composed of sodium alginate, saponin, and guar gum.

### Therapeutic Oil Blends

The present disclosure provides compositions for topical application. In some embodiments, the compositions comprise a therapeutic oil blend comprising one or more cannabinoids and one or more terpenes. In some embodiments, the therapeutic oil blend comprises cannabidiol (CBD). In some embodiments, the therapeutic oil blend comprises one or more terpenes that provide additional desirable properties, e.g., analgesic, anesthetic, anti-inflammatory properties and the like. In some embodiments, methods of formulating the disclosed therapeutic oil blends result in nano-penetrative particle size, non-crystallizing properties, and/or improved homogeneity. Therapeutic oil blends of the present disclosure are discussed in more detail in. PCT/US2021/01911, and U.S. patent application Ser. No. 17/327,234, both of which are incorporated by reference in their entireties for all purposes.

The cannabinoids and terpenes comprised by the therapeutic oil blend are discussed in depth in the foregoing sections, entitled “Cannabinoids” and “Terpenes.”

In some embodiments, the therapeutic oil blend comprises cannabinoids in a weight percentage of about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% by weight of the therapeutic oil blend. In some embodiments, the therapeutic oil blend comprises about 25% to about 75% by weight of a cannabinoid or a mixture of cannabinoids. In some embodiments, the therapeutic oil blend comprises about 30% to about 70% by weight of a cannabinoid or a mixture of cannabinoids. In some embodiments, the therapeutic oil blend comprises a cannabis-derived composition, such as a hemp oil, comprising one or more cannabinoids.

In some embodiments, the therapeutic oil blend of the present disclosure comprises one or more terpenes selected from beta-myrcene, linalool, alpha-pinene, beta-pinene, beta-caryophyllene, caryophyllene oxide, camphor, alpha-humulene, nerolidol, d-limonene, 1-limonene, para-cymene, eugenol, farnesol, geraniol, phytol, menthol, menthone, terpineol, alpha-terpineol, benzaldehyde, hexyl acetate, methyl salicylate, eucalyptol, ocimene, terpinolene, alpha-terpinene, isopulegol, guaicol, alpha-bisabolol, or any combination thereof.

In some embodiments, the therapeutic oil blend comprises about 0.05% to about 80% by weight of a terpene or a mixture thereof. In some embodiments, the therapeutic oil blend comprises about 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, or 95% by weight of a terpene or a mixture thereof. In some embodiments, the therapeutic oil blend comprises about 0.1% to about 50% by weight of a terpene or a mixture thereof. In some embodiments, the therapeutic oil blend comprises about 5% to about 20% by weight of a terpene or a mixture of terpenes. In some embodiments the therapeutic oil blend comprises a total terpene w/w content between 25% and 75%.

### Physical Characteristics of Therapeutic Oil Blend

The present disclosure provides novel therapeutic oil blends, and methods of formulation thereof, that overcome common problems prevalent among typical cannabinoid oil formulations. Existing formulations often encounter problems of large particle size, low absorption, short shelf life, and poor stability. For example, many existing formulations encounter difficulties with separation of constituent ingredients and/or crystallization over time, often on short time scales. This necessitates re-homogenization prior to use, a time-consuming and inconvenient process limiting their use on demand. Furthermore, these problems with existing oil formulations result in their limited ability to be mixed into other compositions and commercial products.

By contrast, in some embodiments, the present therapeutic oil blends have numerous beneficial physical characteristics differentiating them from currently available formulations. These properties may include improved shelf stability, which characteristic includes both non-separation and non-crystallization under typical storage conditions. In some embodiments, the therapeutic oil blend has improved shelf stability compared to existing therapeutic oil blends. In some embodiments, the therapeutic oil blend is liquid at room temperature. In some embodiments, the therapeutic oil blend does not separate and/or crystallize at room temperature in a sealed container for at least 1 week, 2 weeks, 3 weeks, 4 weeks, 5 weeks, 6 weeks, 7 weeks, or 8 weeks. In some embodiments, the therapeutic oil blend does not separate and/or crystallize at room temperature in a sealed container for at least 1 month, 2 months, 3 months, 4 months, 5 months, 6 months, 7 months, or 8 months, 9 months, 10 months, 11 months, or 12 months. In some embodiments, the therapeutic oil blend does not separate and/or crystallize at room temperature in a sealed container for at least 1 year, 2 years, 3 years, 4 years, or 5 years. In embodiments, the therapeutic oil blend has reduced or no: separation, discoloration, or change in consistency at 37° C. for a period of three months. In embodiments, the therapeutic oil blend that comprises an oil-in-water emulsion composition has reduced or no: separation, discoloration, or change in consistency at 37° C. for a period of 1 month, 2 months, 3 months, 4 months, 5 months, 6 months, 7 months, or 8 months, 9 months, 10 months, 11 months, or 12 months. Any one of separation, discoloration, or change in consistency can be evaluated using an in vitro assay such as microscopy, time-lapse microscopy, chromatography, inverse capillary velocity, coalescence assay, and the like. In some embodiments, the container is not sealed or is not airtight and the therapeutic oil blend still does not separate and/or crystallize for the time periods recited in the foregoing embodiments.

In some embodiments, the improved shelf life of the present therapeutic oil blend is characterized by its improved shelf half-life, as measured through crystallization, separation, or degradation of therapeutic oil blend. The compositions described herein can have a shelf half-life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 240, 270, 300, 330, or 360 days. In some cases, the compositions described herein can have a shelf half-life of at least about 1, 2, 3, 4, or 5 years. The therapeutic oil blend disclosed herein can be characterized by a cannabinoid degradation rate at an ambient temperature of at least 20° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than the degradation rate of a non nano-penetrative cannabinoid composition.

The therapeutic oil blend may have a small particle size, which may facilitate dermal absorption. In some embodiments, the therapeutic oil blend has a mean particle size of 20-60 nm. In some embodiments, the cannabinoid component of the therapeutic oil blend is suspended or contained within phases having a mean particle size of 20-60 nm. In some embodiments, the terpenes of the therapeutic oil blend are suspended or contained within phases having a mean particle size of 20-60 nm. In some embodiments, the therapeutic oil blend is nano-penetrative.

In some embodiments, the small particle size leads to increased bioavailability and/or increased bioactivity. The therapeutic oil blends described herein, when administered to a subject, can have improved bioavailability, bioactivity, or both. Bioavailability is the fraction of an administered dosage of unchanged compound that reaches systemic circulation. In some embodiments, the therapeutic oil blend disclosed herein can be characterized by a bioavailability in a subject of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10.0 or more times that of a non-nano-penetrative cannabinoid composition (e.g., a composition lacking the therapeutic oil blend). In some embodiments, the therapeutic oil blend disclosed herein can be characterized by a bioavailability in a subject of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, including all ranges and subranges therebetween. Bioactivity, or biological activity, is the activity exerted by the active ingredient or ingredients in a composition. In some embodiments, the therapeutic oil blend disclosed herein can be characterized by a bioactivity in a subject of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10.0 or more times that of non-nano-penetrative cannabinoid composition.

In embodiments, the therapeutic oil blend is thermostable. In embodiments, an oil-in-water emulsion composition comprising the therapeutic oil blend is thermostable. Thermostability can be determined by evaluating at least one of: separation, discoloration, or consistency, of the composition (e.g. oil-in-water emulsion) at 45° C. for a period of three months. Thermostability can be determined by evaluating at least one of: separation, discoloration, or consistency, of the composition (e.g. oil-in-water emulsion) at 45° C. for a period of 1 month, 2 month, 3 month, 4 month, 5 month, 6 month, 7 month, 8 month, 9 month, 10 month, 11 month, or up to 12 months. In embodiments, thermostability can be determined by evaluating at least one of: separation, discoloration, or consistency, of the composition (e.g. oil-in-water emulsion) at 35° C.-55° C. for a period of 1 month, 2 month, 3 month, 4 month, 5 month, 6 month, 7 month, 8 month, 9 month, 10 month, 11 month, or up to 12 months.

### Formulation of Therapeutic Oil Blend

The therapeutic oil blends of the present disclosure may be formulated according to known methods of oil blend preparation. Methods may include heating, cooling, mixing, serial addition, serial dilution, emulsification, homogenization, dissolution, titration, and the like.

In some embodiments, the therapeutic oil blends may be emulsified with one or more emulsifiers disclosed herein. That is, in some embodiments, the therapeutic oil blends of the present disclosure form the oil phase of the presently claimed oil in water emulsions. In some embodiments, the therapeutic oil blend may be emulsified into the presently claimed lotions according to the methods set forth in U.S. Pat. Pub. No. 20190298683, herein incorporated by reference in its entirety.

In some embodiments, the therapeutic oil blend is formulated through the use of sonication. In some embodiments, the therapeutic oil blend is sonicated with an ultrasonic homogenizer. The sonication times, temperatures, amplitude, and frequency may be tuned to obtain smaller particle sizes. The total amount of energy (E) delivered to a suspension not only depends on the applied power (P) but also on the total amount of time (t) that the suspension is subject to the ultrasonic treatment: E=P×t. In some embodiments the cannabinoid-containing oil (also referred to herein as the cannabinoid oil), e.g. hemp oil, is heated prior to initial sonication. The formation of cavitation bubbles will increase the temperature of the sonicated medium, such that temperature limits should be set throughout sonication to ensure a reasonable temperature range during formulation. during this process. Calorimetry can be used to measure the effective acoustic energy delivered to a sonicated liquid in a device independent manner. The method is based on the measurement of the temperature increase in a liquid medium over time as a result of cavitation induced in a liquid by an immersed ultrasound probe. At a given device output power setting, the temperature increase in the liquid is recorded over time and the effective delivered power can be calculated using the following equation: P=((dT/dt)MCp) where P is the delivered acoustic power (W), T and t are temperature (K) and time (s), respectively, Cp is the specific heat of the liquid (J/g·K) and M is the mass of liquid (g). For cooling the mixture throughout the sonication process, ice water baths may be employed. The sonicated cannabinoid comprising oil may be brought down to room temperature before homogenizing the other terpenes into the cannabinoid comprising oil. Foaming can be avoided through the use of lower power settings following the addition of any surfactant.

In some embodiments, the formulation of the therapeutic oil blend begins with providing a cannabinoid-containing oil, referred to as a cannabinoid oil. In some embodiments, the cannabinoid oil is substantially pure, e.g., substantially free of other ingredients. In some embodiments, the cannabinoid oil may consist almost exclusively of the cannabinoids of interest without other primary ingredients. In some embodiments, the cannabinoid oil is a hemp oil. In some embodiments, the cannabinoid oil is a CBD oil.

In some embodiments, the first step is to heat the cannabinoid oil. In some embodiments, the cannabinoid oil is heated to about 40° C., 45° C., 50° C., 55° C., 60° C., 65° C., 70° C., 75° C., or 80° C. to fully dissolve any cannabinoids that may have solidified during transport or storage. In some embodiments, the oil is heated to a temperature above 40° C. In some embodiments, the cannabinoid oil is heated to about 60° C.

In some embodiments, the next step is to sonicate the heated cannabinoid oil. In some embodiments, the cannabinoid is sonicated for a period on the order of hours of active sonication time. In some embodiments, the cannabinoid oil is sonicated for about 1, 2, 3, 4, or 5 hours of active sonication time. In some embodiments, the cannabinoid oil is sonicated for about 2 hours of active sonication time. In some embodiments, the cannabinoid oil is sonicated until the desired level of cavitation has been achieved. In embodiments, a level of cavitation is determined by microscopy, based on the accumulation of cavitation bubbles.

In some embodiments, the sonication is carried out between 15-25 kHz and at a power level of between 1500 and 2000 W. In some embodiments, the sonication is carried out at about 22 kHz and/or at about 1800 W

In some embodiments, the sonication step can raise the temperature and cause the cannabinoid oil to overheat. In some embodiments, the present disclosure teaches keeping the oil below 80° C.

In some embodiments, the sonication alternates between on and off periods, e.g. 10 seconds on and 10 seconds off.

In some embodiments, the next step is to cool the cannabinoid oil. In some embodiments, the cannabinoid oil is brought down to room temperature, or around 15° C.-30° C. In some embodiments, the sonicated cannabinoid oil is brought to 23° C.

In some embodiments, the next step is to add terpenes to the sonicated cannabinoid oil. In some embodiments, the present disclosure teaches homogenizing the terpenes into the cannabinoid oil via sonication.

In some embodiments, eugenol is the first terpene to be added or one of the first terpenes to be added to the sonicated cannabinoid oil. In some embodiments, eugenol is chilled prior to addition. In some embodiments, eugenol is added slowly during active sonication. In some embodiments, sonication is maintained at a level of about 15-25 kHz, alternating between on and off periods. In some embodiments, sonication is carried out at around 20 kHz. In some embodiments, the power level of sonication is set to about 500-1000 W. In some embodiments, the homogenization of the cannabinoid oil and eugenol is carried out for 5-30 minutes, e.g., 10 minutes of active sonication time. In some embodiments, the sonication alternates on a 30 s/5 s interval of on/off.

In some embodiments, d-limonene is added second to the therapeutic oil blend. D-limonene may be used to increase the dermal absorption characteristics of the therapeutic oil blend when added in this order. In some embodiments, d-limonene is added slowly during active sonication. In some embodiments, sonication is maintained at a level of about 15-25 kHz, alternating between on and off periods. In some embodiments, sonication is carried out at around 22 kHz. In some embodiments, the power level of sonication is set to about 1500-2000 W. In some embodiments, the homogenization of the cannabinoid oil mixture and d-limonene is carried out for 5-30 minutes, e.g., 15 minutes of active sonication time. In some embodiments, the sonication alternates on a 60 s/17 s interval of on/off.

In some embodiments, all additional terpenes, including menthol and menthone, are adding during a subsequent step of sonication. In some embodiments, the additional ingredients are added slowly during active sonication. In some embodiments, sonication is maintained at a level of about 15-25 kHz, alternating between on and off periods. In some embodiments, sonication is carried out at around 19 kHz. In some embodiments, the power level of sonication is set to about 100-500 W. In some embodiments, the homogenization of the cannabinoid oil mixture and the additional ingredients is carried out for 5-30 minutes, e.g., 15 minutes of active sonication time. In some embodiments, the sonication alternates on a 5 s/15 s interval of on/off.

In some embodiments, after the addition of each of the constituents, the cannabinoid oil mixture is sonicated for an additional period of time to produce the fully homogenized therapeutic oil blend with the desired average particle size. In some embodiments, sonication is maintained at a level of about 15-25 kHz, alternating between on and off periods. In some embodiments, sonication is carried out at around 22 kHz. In some embodiments, the power level of sonication is set to about 1500-2000 W. In some embodiments, the homogenization of the cannabinoid oil mixture is carried out for 20-60 minutes, e.g., 30 minutes of active sonication time. In some embodiments, the sonication alternates on a 45 s/120 s interval of on/off.

Throughout the above steps, in some embodiments, the present disclosure teaches regulating the temperature of the oils and mixtures throughout the homogenization process leading to the formation of the therapeutic oil blend. In some embodiments, the present disclosure teaches keeping the temperature of the oils and mixtures below a pre-determined set point. In some embodiments, the set point may be fixed throughout the homogenization process. In some embodiments, the set point may vary in different steps.

In some embodiments, the set point is between 30° C.-80° C., i.e., the mixture is maintained below this temperature. In embodiments, the set point is from about 30° C., 32° C., 34° C., 36° C., 38° C., 40° C., 42° C., 44° C., 46° C., 48° C., 50° C., 52° C., 54° C., 56° C., 58° C., 60° C., 62° C., 64° C., 66° C., 68° C., 70° C., 72° C., 74° C., 76° C., 78° C., or up to about 80° C., including all ranges and subranges therein. In some embodiments, the set point is 60° C. In some embodiments, the set point is 40° C. Temperature may be regulated by a variety of means, e.g., ice bath, water bath, coolant, fans, refrigeration, chilling, resting to allow ambient temperature to return, etc.

In addition, measures may be taken to avoid foaming after addition of surfactants (e.g., terpenes with surfactant properties). In some embodiments, long rest periods are employed to avoid foaming in the presence of surfactants. Sonication may also be employed in pulse mode to help avoid foaming. In some embodiments, sonication is generally carried out in alternating periods of on and off, e.g., 10/10, 30/5, 60/20, 5/15, 45/120 in terms of on seconds/off seconds. The present methods are not limited to any particular alternation of sonication or particular period of time. The times may be determined by one of skill in the art in order to maintain temperature below the set point, avoid foaming, increase homogenization, and the like. Longer rest periods may be employed to maintain a lower temperature and/or decrease foaming. The sonication power and frequency may be adjusted as well to improve one or more of these parameters.

The foregoing steps may be used to create a therapeutic oil blend of the present disclosure with small particle size (20-60 nm average), nano-penetrative qualities, and/or non-crystallizing qualities.

### Exemplary Therapeutic Oil Blends

The present disclosure provides methods of formulating therapeutic oil blends comprising at least one cannabinoid and at least one terpene.

In some embodiments, the cannabinoid is Cannabidiol. In some embodiments, the cannabinoid is a Cannabidiol-related cannabinoid.

In some embodiments, the therapeutic oil blend comprises a terpene selected from the list consisting of bisabolol, borneol, caryophyllene, carene, camphene, camphor, cineol, citronellal, eucalyptol, eugenol, geraniol, guaiol, humulene, isopropyltoluene, isopulegol, linalool, d-limonene, menthol, menthone, beta-myrcene, nerolidol, ocimene, alpha-pinene, beta-pinene, phytol, pulegone, alpha-terpinene, gamma-terpinene, terpinolene, and thymol. In some embodiments, the therapeutic oil blend comprises eugenol. In some embodiments, the therapeutic oil blend comprises menthol. In some embodiments, the therapeutic oil blend comprises menthone. In some embodiments, the therapeutic oil blend comprises d-limonene. In some embodiments, the therapeutic oil blend comprises eugenol, menthol, menthone, and d-limonene. In some embodiments, the therapeutic oil blend comprises eugenol, menthol, menthone, and d-limonene and at least one more, two more, three more, or four more terpenes selected from the list consisting of bisabolol, borneol, caryophyllene, carene, camphene, camphor, cineol, citronellal, eucalyptol, geraniol, guaiol, humulene, isopropyltoluene, isopulegol, linalool, beta-myrcene, nerolidol, ocimene, alpha-pinene, beta-pinene, phytol, pulegone, alpha-terpinene, gamma-terpinene, terpinolene, and thymol.

### Methods of Use of Compositions of the Disclosure

The compositions of the present disclosure may be used in the treatment of numerous conditions. In some embodiments, the compositions may be administered to improve one or more physical attributes associated with a condition.

In some embodiments, the compositions may be administered to improve one or more physical attributes in the absence of a medical condition or unrelated to a medical condition. For example, the compositions may be administered to improve sense of wellbeing, recovery after physical exertion, skin quality, muscle tension, or any of a number of physical attributes.

Subjects of the present disclosure can include humans and other animals, such as pets (e.g., dogs, cats, birds, small mammals, snakes) and livestock or farm animals (e.g., cows, pigs, horses, sheep, chickens). Compositions of the present disclosure can be useful for both human and veterinary applications.

### Exemplary Diseases and Conditions

In embodiments, any of the compositions provided herein can be used to reduce, treat, and/or prevent a disease or condition. In embodiments, a composition is used preventively in a healthy subject. In some embodiments, the compositions may be used in the treatment of pain. Examples of pain include muscle pain, neuropathic pain, neurogenic pain, back pain, migraine, headache, facial pain, endometriosis, neuropathic pain associated with post-herpetic neuralgia, diabetic neuropathy, post-episiotomy pain, joint pain, musculoskeletal pain, trigeminal neuralgia, chronic pain, and the like. The compositions of the present disclosure may be used to mediate pain perception and inflammation, and indicate potential in the topical symptomatic treatment of the following conditions: neuropathic pain, burning feet syndrome, diabetic foot neuropathy, chronic low back pain, fibromyalgia syndrome, neck pain, post herpetic neuralgia, pain and inflammation of arthritis (hands, knees, joints); rheumatoid arthritis and osteoarthritis, trigeminal neuralgia (TN), pudendal neuropathy and pudendal nerve entrapment (PNE), sciatica pain, muscle strains, tendon injuries, plantar fasciitis, herniated disks; phantom limb pain following amputation, and dermatological disorders like psoriasis, eczema, dermatitis.

In some embodiments, a composition of the present disclosure may be used irrespective of the presence or absence of a medical condition. In some embodiments, a composition may be used to improve one or more aspects of daily life. In some embodiments, a composition may be used for the treatment of stress, for improving mood, for increasing relaxation, or for promoting a sense of well-being. In some embodiments, a composition reduces the symptoms of sore muscles, muscle aches, joint pain, muscle cramping, and/or stiffness.

In embodiments, pain is evaluated using a pain assessment scale. Exemplary scales include but are not limited to Numerical Rating Scale (NRS), Visual Analog Scale (VAS), Defense and Veterans Pain Rating Scale (DVPRS), Adult Non-Verbal Pain Scale (NVPS), Pain Assessment in Advanced Dementia Scale (PAINAD), Behavioral Pain Scale (BPS), Critical-Care Observation Tool (CPOT), and combinations thereof. In embodiments, pain may also be evaluated via interview with a subject. In embodiments, a composition of the present disclosure may reduce pain by at least about 1-10 marks on a pain assessment scale. In embodiments, a composition of the present disclosure may reduce pain by at least about 1, 2, 3, 4, 5, 6, 7, 8, 9, or up to 10 marks on a pain assessment scale.

In embodiments, mood and relaxation may also be evaluated using a scale. In embodiments, a composition of the present disclosure may increase mood and/or relaxation by at least about 1-10 marks on an assessment scale. In embodiments, a composition of the present disclosure may increase mood and/or relaxation by at least about 1, 2, 3, 4, 5, 6, 7, 8, 9, or up to 10 marks on an assessment scale.

In some embodiments, a composition of the present disclosure, e.g., a topical formulation, may be used to improve one or more aspects of skin health, quality, or maintenance. For example, a composition may be used to improve skin hydration, appearance, dryness, tautness, tension, oiliness, texture, or smoothness.

In embodiments, a skin assessment is performed to evaluate improvements in the skin. A skin assessment may comprise evaluating any one of: temperature moisture, turgor, integrity (e.g. flake, rash, wound, scarring, callus, cellulitis, discoloration, and combinations), color, sensation (e.g. numbness, burning, itching, pain, and the like. In embodiments, a physician and/or nurse may perform a skin assessment via visual examination and/or interview with a subject.

In some embodiments, a composition of the present disclosure may be used to improve one or more aspects of physical health related to routine physical fitness. For example, a composition may be used to improve muscle regeneration, muscular discomfort, joint discomfort, soreness, muscle aches, muscle strain, or muscle exhaustion related to physical activity or exercise. In some embodiments, a composition may be applied (in the case of a topical formulation) following a period of physical exertion to improve one or more aspects of the recovery experience, including but not limited to recovery duration, comfort, stiffness, soreness, and muscle/joint pain. Any of the aforementioned aspects of physical health related to routine physical fitness may be evaluated by interview with a subject by a physician, therapist, physical trainer, and the like.

### Administration

For the purposes of administration, the present compositions may be formulated in a variety of dosage forms. The term “dosage form” denotes any form of the formulation that contains an amount of a cannabinoid or of a mixture of cannabinoids and a terpene or a mixture of terpenes sufficient to achieve at least a partial therapeutic effect with a single or repeat administration. In some embodiments, the dosage form is a topical dosage form. In some embodiments, the dosage form is one of those described in the foregoing sections, e.g., an oil, a massage oil, a lotion, a gel, a salve, a body balm, and the like. In some embodiments, the dosage form is a lotion or a body balm.

Compositions can be formulated in forms including but not limited to liquid, gel, semi-solid, and solid. Compositions disclosed herein can further be processed into forms including but not limited to solids, liquids, suspensions, gels, lotions, balms, and other forms discussed in this disclosure. In embodiments, a composition comprises a phase selected from the group consisting of: liquid, gel, semi-solid, solid, and combinations thereof. In embodiments, a composition is a liquid. In embodiments, a composition is a gel. In embodiments, a composition is semi-solid.

Cannabinoids, terpenes and flavonoids may be lipophilic and/or have low solubility in hydrophilic biocompatible matrix materials. One method for obtaining desirable dosage forms comprising lipophilic substances and hydrophilic biocompatible matrix substances is to encapsulate or disperse lipophilic substances in the hydrophilic matrix using additives or modifiers which provide an environment for stable oil-in-water emulsions, micelles, liposomes or other complex phase equilibrium modified compositions. Exemplary techniques, modifiers and additives are described, e.g., in U.S. Publication No. 20190321330, incorporated by reference herein in its entirety.

In some embodiments, the composition is for sale in unit form, e.g., in a bottle, tube, tub, or other container. In some embodiments, the unit comprises at least about 50, 100, 150, 200, 250, 300, 350, 400, 450, 500, 550, 600, 650, 700, 750, 800, 850, 900, 950, 1000, 1100, or 1200 mg, or any ranges or subranges therebetween, of a cannabinoid or a mixture thereof. In some embodiments, the unit comprises at least about 150 mg of a cannabinoid or a mixture thereof. In some embodiments, the unit comprises at least about 300 mg of a cannabinoid or a mixture thereof. Each possibility represents a separate embodiment of the disclosure.

In some embodiments, an effective amount of a composition is administered to a subject. The term “effective amount” or “therapeutically effective amount” refers to that amount of a composition described herein that is sufficient to effect the intended application including but not limited to a reduction in oxidative stress in a cell and/or disease treatment in a subject. The therapeutically effective amount may vary depending upon) the subject and condition being treated, e.g., the weight and age of the subject, the severity of the disease condition, the manner of administration and the like, which can readily be determined by one of ordinary skill in the art. The term also applies to a dose that will induce a particular response in target cells, e.g., reduction in oxidative stress, reduction in pain, anesthesia effect, analgesic effect, etc. The specific dose will vary depending on the particular formulation of the cannabinoid composition, the dosing regimen to be followed, whether it is administered in combination with other compounds, timing of administration, the tissue to which it is administered, route of administration and the physical delivery system in which it is carried.

Any of the subject compositions can be provided in a unit dosage form. A unit dosage is an amount of a compound, such as a cannabinoid compound delivered alone or in combination with other components, which is to be administered to a subject at or about one time point. Other components which can be included with a unit dosage include but are not limited to cosmetics, food carriers, food bars, baked goods, dairy products, oils, beverages, solid dosages (e.g., tablets), or liquid dosages. A unit dosage of a cannabinoid compound can be about 10, 20, 30, 40, 50, 60, 70, 80, 90, 100, 125, 150, 175, 200, 250, 300, 350, 400, 450, 500, 600, 700, 800, 900, 1000, 1100, 1200, 1300, 1400, 1500, 1600, 1700, 1800, 1900, 2000 or more milligrams (mg). A unit dosage of a cannabinoid compound can be at least about 10, 20, 30, 40, 50, 60, 70, 80, 90, 100, 125, 150, 175, 200, 250, 300, 350, 400, 450, 500, 600, 700, 800, 900, 1000, 1100, 1200, 1300, 1400, 1500, 1600, 1700, 1800, 1900, 2000 or more milligrams (mg). A unit dosage of a cannabinoid compound can be at most about 2000, 1900, 1800, 1700, 1600, 1500, 1400, 1300, 1200, 1100, 1000, 900, 800, 700, 600, 500, 450, 400, 350, 300, 250, 200, 175, 150, 125, 100, 90, 80, 70, 60, 50, 40, 30, 20, 10, or less milligrams (mg). A unit dosage can be an hourly dosage. A unit dosage can be a daily dosage. A unit dosage can provide about 1/24, 1/12, ⅛, ⅙, ¼, ⅓, ½, or all of a daily dosage of one or more cannabinoids for a subject. A unit dosage can take the form of a tablet, gel, liquid, food product, food bar, container of liquid of defined volume, or other forms described herein, packaged for one-time consumption or administration.

The amount of the composition of the subject method administered will be dependent on the subject being treated, the severity of disorder or condition, the rate of administration, the disposition of the composition and encapsulated cannabinoid compound. An effective dosage is in the range of about 0.1 mg to about 2000 mg per kg body weight per day. For example, for a 70 kg human, this may amount to about 7 mg/day to about 1.75 g/day. In some instances, dosage levels below the lower limit of the aforesaid range may be more than adequate, while in other case still larger doses may be employed without causing any harmful side effects, e.g., by dividing such larger doses in several small doses for administration throughout the day. Dosages may be administered over time periods of hours, days, weeks, or months.

### Kits

The disclosure also comprises a kit that includes any of the compositions provided herein (e.g. lotion and/or balms). In embodiments, the kit comprises containers suitable for applying to the skin or epithelium of a subject. Exemplary containers include but are not limited to: tubes, tubs, pumps, syringes, pots, bottles, buckets, and the like.

Kits for administering the compositions of the inventions, either under direction of a health care professional or by the patient or subject, may also include a custom applicator suitable for that purpose. In embodiments, kits may also comprise instructions for use thereof.

The aspects and embodiments provided herein have been described in an illustrative manner, and it is to be understood that the terminology used is intended to be in the nature of words of description rather than of limitation.

Many modifications and variations are possible in light of the above teachings. It is, therefore, to be understood that within the scope of the appended claims, the disclosure can be practiced otherwise than as specifically described.

The present disclosure is illustrated in detail below with reference to examples, but is not to be construed as being limited thereto.

Citation of any document herein is not intended as an admission that such document is pertinent prior art, or considered material to the patentability of any claim of the present disclosure. Any statement as to content or a date of any document is based on the information available to applicant at the time of filing and does not constitute an admission as to the correctness of such a statement.

### EXAMPLES

### Example 1: Therapeutic Oil Blend Formulation Process Via Sonication

This example describes the production of therapeutic oil blends that can be used in later formulating the oil-in-water emulsions of the present disclosure. In this example, CBD was used. A therapeutic oil blend comprising Cannabidiol, eugenol, menthol, menthone, and d-limonene was prepared using an ultrasonic homogenizer (1800 W, 19-22 kHz). The cannabidiol-related cannabinoid was provided in the form of a high purity, high concentration cannabidiol containing hemp oil. The hemp oil was heated to 60° C. and sonicated for two hours at maximum power, alternating between sonication and rest periods at every 10 seconds. To this was added chilled eugenol during a further sonication cycle of ten minutes alternating between sonication and rest time. To this mixture was added chilled d-limonene during a further sonication cycle of fifteen minutes alternating between sonication and rest time. Camphor, eucalyptol, a-pinene, b-pinene, b-myrcene, a-terpinene, y-terpinene, and linalool were mixed at room temperature, allowing any solid ingredients to dissolve, and added during another sonication cycle of 20 minutes alternating between sonication and rest time. The therapeutic oil blend was then cooled and set to run for two hours alternating between sonication and rest time. The resulting therapeutic oil blend had an average particle size of 20-60 nm and was shelf stable without crystallization for a period of at least 30 days, time point limited only by observation length. In contrast, the control mixture of the ingredients without sonication crystallized in 24 hours, had a sticky texture, and did not absorb well into the skin.

### Example 2: Therapeutic Oil Blend Formulation Process Via Sonication

Additional cannabinoids will be formulated according to Example 1 to produce alternative therapeutic oil blends. For example, a therapeutic oil blend comprising a cannabidiol-related cannabinoid, eugenol, menthol, menthone, and d-limonene is prepared using an ultrasonic homogenizer (1800 W, 19-22 kHz). The cannabidiol-related cannabinoid is provided in the form of a high purity, high concentration cannabidiol-related cannabinoid containing hemp oil. The hemp oil is heated to 60° C. and sonicated for two hours at maximum power, alternating between sonication and rest periods at every 10 seconds. To this is added chilled eugenol during a further sonication cycle of ten minutes alternating between sonication and rest time. To this mixture is added chilled d-limonene during a further sonication cycle of fifteen minutes alternating between sonication and rest time. Remaining terpenes are mixed at room temperature and added during another sonication cycle of 20 minutes alternating between sonication and rest time. The therapeutic oil blend is then cooled and set to run for two hours alternating between sonication and rest time to ensure complete mixing. The resulting therapeutic oil blend has an average particle size of 20-60 nm and is shelf stable without crystallization for a period of at least 30 days.

### Example 3: Illustrative Compositions of the Present Disclosure

The following lotions and balms are exemplary compositions of the present disclosure having the desired characteristics, including thermostability and consistency.

### Preparation of Exemplary Lotions

Lotions of the present disclosure are prepared according to the following general progression: water phase→emsulfiers→oil phase→ethanol phase. Throughout the formulation process, the temperature of the composition is maintained below 35° C. (e.g., below 30° C.). Cooling was performed by jacketed cooling or water bath. The formulation process of lotions of some embodiments of the present disclosure is as follows:

1.) Combined the ingredients in the water phase together and slowly mixed with a rotor stator homogenizer until combined.

2.) Added in the saponin and slowly mixed until fully dissolved.

3.) With the rotor stator homogenizer on max power (6000 krpm-15 krpm), slowly added in the sodium alginate and the guar gum together, adding the full amount over 20-30 seconds. This mix started to foam and thicken, thus facilitating homogenization. Mixing continued until smooth.

4.) The oil phase ingredients were then combined together in a separate container and then slowly added into the thickened water phase.

5.) The oil phase was added at a slow constant rate at the input of the homogenizer work area.

6.) Once the oil phase was added, the ingredients were mixed until smooth. At this stage, the mixture resembled thick meringue.

7.) Added in the ethanol phase. At this point, the mixture appeared clumped throughout.

8.) Allowed the homogenizer to work, and the mixture started to form a custard-like consistency again. Allowed the mixture to continue to homogenize until it was light, airy, and smooth.

### Ingredients and Characterization of Exemplary Compositions

The ingredients for exemplary lotion A are listed in Table 1.

TABLE 1
Exemplary lotion A

| | Percent | Mass |
|---|---|---|
| Ingredient | Composition | (mg) |
| Purified Water | 50.87% | 28995.33 |
| Ethyl Alcohol, Organic | 20.0% | 11400 |
| Olive Oil, Extra Virgin, Organic | 5.6% | 3192 |
| Sunflower Oil, High Oleic Virgin, Organic | 5.6% | 3192 |
| Hemp Seed Oil, Golden Virgin, Organic | 0.6% | 342 |
| Glycerin USP, Organic | 4.68% | 2666.46 |
| Lavender Bulgarian, Organic | 1.5% | 855 |
| Menthol Crystals, Organic | 1.5% | 855 |
| Ravintsara (Camphor), Organic | 1.5% | 855 |
| Eucalyptus Oil, Organic | 1.5% | 855 |
| Arnica CO2 Extract Blend, Organic | 1.0% | 570 |
| Black Pepper Oil, Organic | 1.0% | 570 |
| CBD Extract, Organic | 1.05% | 600 |
| Quillaja Saponin | 1.0% | 570 |
| Sodium Alginate | 1.0% | 570 |
| Turmeric CO2 Extract, Organic | 1.0% | 570 |
| Sea Buckthorn Oil, Organic | 0.3% | 171 |
| Guar Gum, 3500, Organic | 0.3% | 171 |
| Total: | 100% | 57000 |

Lotion A has a water phase comprising 55.55% of the composition; an oil phase of 22.15%; and an alcohol phase of 20%, with percentages given by mass. The remaining constituents, making up 2.3% of the lotion are the saponin, sodium alginate, and guar gum. The water phase comprises purified water and glycerin. The oil phase comprises olive oil, sunflower oil, hemp seed oil, lavender oil, menthol, camphor, eucalyptus oil, CO₂-extracted arnica oil, black pepper oil, a CBD extract (i.e., a therapeutic oil blend as described in the Therapeutic Oil Blend section herein), CO₂-extracted turmeric oil, and sea buckthorn oil. The alcohol phase comprises ethyl alcohol. The ingredient list in Table 1 makes up 2 oz of this composition, such that the density of the composition is 0.96 g/cm³. Lotion A comprises 150 mg of CBD per ounce. Lotion A was determined to have an estimated shelf life and thermostability of 8 months at room temperature, i.e., temperatures less than 80° F.

Lotion A is suitable for use as a general moisturizer and is also useful for improving the symptoms of sore muscles, muscle aches, joint pain, muscle cramping, and stiffness, among other conditions. Lotion A is also suitable for use in sports treatments, e.g. sports massage.

The ingredients for exemplary Lotion B are listed in Table 2.

TABLE 2
Exemplary Lotion B

| | Percent | Mass |
|---|---|---|
| Ingredient | Composition | (mg) |
| Purified Water | 42.77% | 24377 |
| Ethyl Alcohol, Organic | 20.0% | 11400 |
| Olive Oil, Extra Virgin, Organic | 5.6% | 3192 |
| Sunflower Oil, High Oleic Virgin, Organic | 5.6% | 3192 |
| Hemp Seed Oil, Golden Virgin, Organic | 5.6% | 3192 |
| Glycerin USP, Organic | 4.68% | 2668 |
| Australian Tea Tree Oil Organic | 1.0% | 570 |
| Arnica, CO2 Extract Blend, Organic | 1.0% | 570 |
| Lemon Balm Extract, Organic | 3.0% | 1710 |
| Eucalyptus Oil, Radiata, Organic | 2.0% | 1140 |
| Rose Geranium Oil, Organic | 1.1% | 627 |
| CBD Extract, Organic | 1.05% | 600 |
| Quillaja Saponin | 1.0% | 570 |
| Chamomile Extract, Organic | 1.0% | 570 |
| Sodium Alginate | 1.0% | 570 |
| Guar Gum, 3500, Organic | 0.3% | 171 |
| Sea Buckthorn Oil, Organic | 0.3% | 171 |
| Peppermint Oil, Piperita, Organic | 3.0% | 1710 |
| Total: | 100% | 57000 |

Lotion B has a water phase of 47.45%, an oil phase of 26.25%, and an alcohol phase of 24%, with percentages given by mass. The remaining constituents, making up 2.3% of the lotion are the saponin, sodium alginate, and guar gum. The other ingredients fall into the same phases as in exemplary lotion A. In addition, the tea tree oil, rose geranium oil, and peppermint oil are in the oil phase; and the lemon balm extract and the chamomile extract are in the alcohol phase. The ingredient list in Table 1 makes up 2 oz of this composition, such that the density of the composition is 0.96 g/cm³. Lotion B comprises 150 mg of CBD per ounce. Lotion B was determined to have an estimated shelf life and thermostability of 8 months at room temperature, i.e., temperatures less than 80° F.

Lotion B is suitable for use as a general moisturizer and is also useful for improving the symptoms of sore muscles, muscle aches, joint pain, muscle cramping, and stiffness, among other conditions. Lotion B is also suitable for use in sports treatments, e.g. sports massage.

In contrast to the commercially available Dr. Bronner's organic lotion, which only has a viscosity around 700-800 cps, exemplary lotions A and B have viscosities in the range of 5000-6500 cps. As such, the present compositions provide an organic lotion with a viscosity several fold higher than what is commercially available.

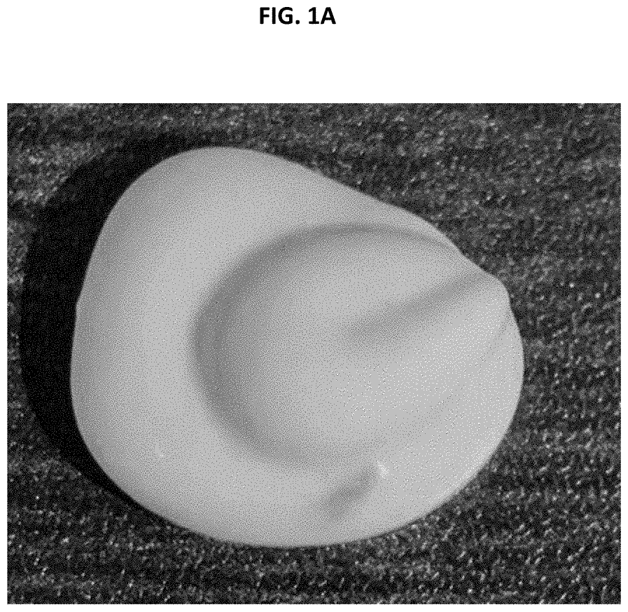

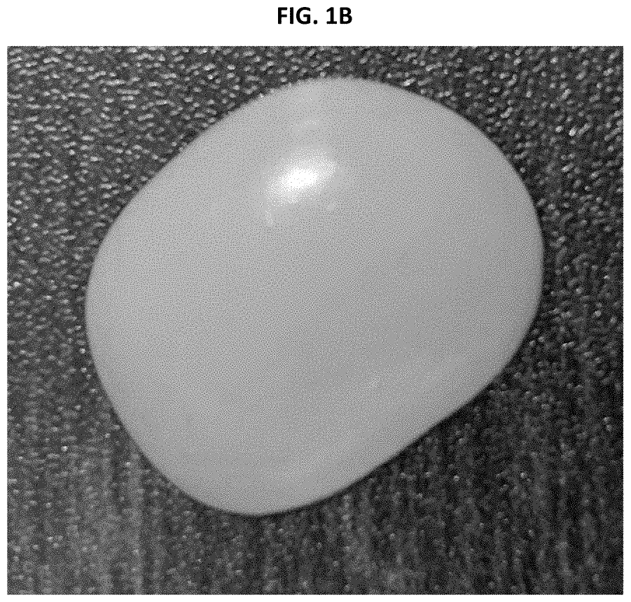

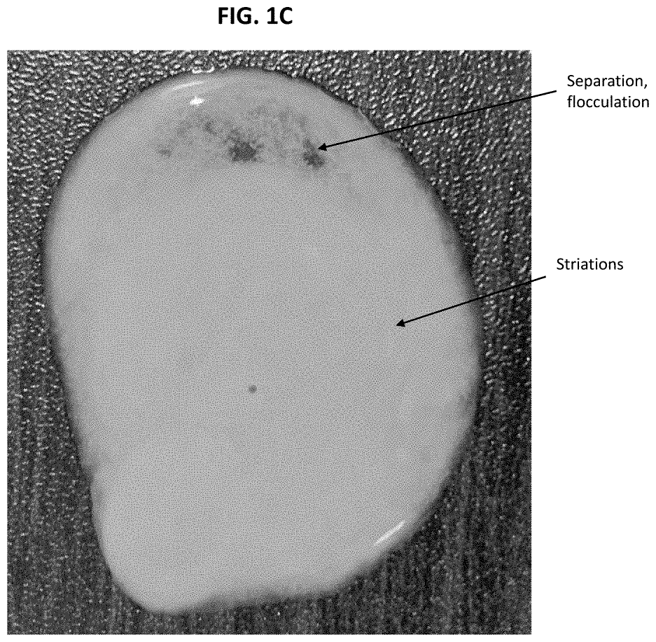

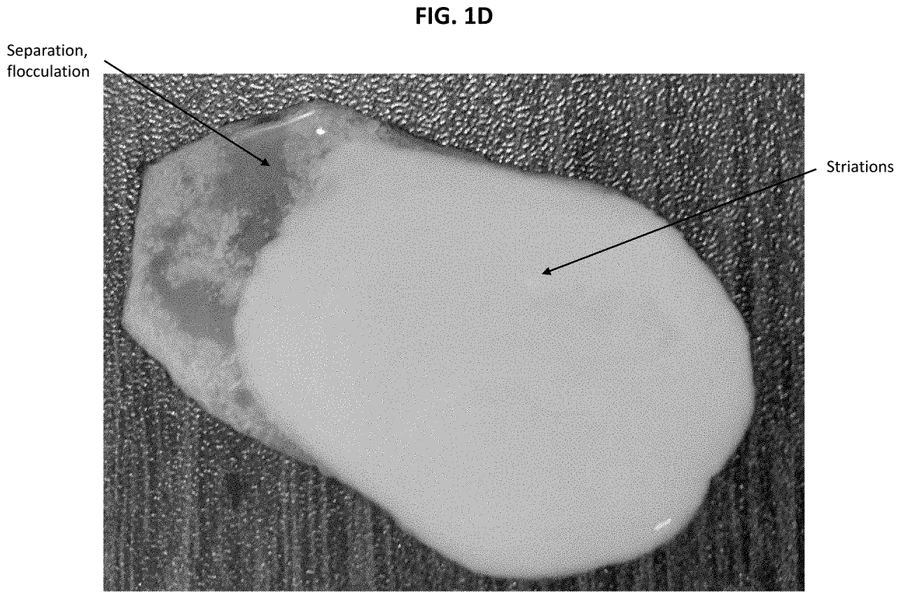

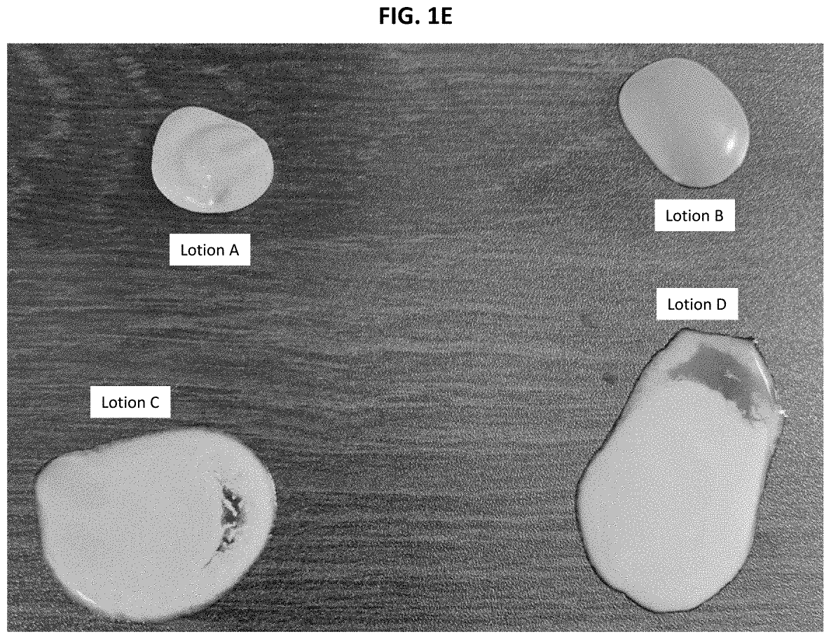

FIGS. 1A and 1B show images of Lotions A and B, respectively, demonstrating that they have the higher viscosity typical of commercial lotions.

The ingredients for an exemplary Balm A are listed in Table 3.

TABLE 3
Exemplary Balm A

| | Percent | Mass |
|---|---|---|
| Ingredient | Composition | (mg) |
| Shea Nut Butter, White Refined, Organic | 30.50% | 8540 |
| Beeswax, Yellow, Organic | 30.00% | 8400 |
| MCT Oil, Unflavored, Organic | 26.93% | 7540 |
| Turmeric CO2 Extract, Organic | 4.00% | 1120 |
| CBD Extract, Organic | 3.57% | 1000 |
| Arnica, CO2 Extract Blend, Organic | 2.00% | 560 |
| Menthol Crystals, Organic | 1.00% | 280 |
| Ravintsara (Camphor), Organic | 1.00% | 280 |
| Eucalyptus Oil, Radiata, Organic | 1.00% | 280 |
| Total: | 100% | 28000 |

This exemplary balm has a density of 0.95 g/cm³, as 28000 mg form 1 oz of product. Balm A comprises 250 mg CBD per ounce. Balm A was determined to have an estimated shelf life and thermostability of 2 years at room temperature, i.e., temperatures less than 80° F.

Balm A may be used in the amelioration of symptoms of sore muscles, muscle aches, joint pain, muscle cramping, and stiffness, among other conditions. The beeswax and shea butter constituents of the balm, in combination with a high potency of essential oils and CBD oil, allows for an extended release of sensation, as the potent ingredients may be embedded in the high viscosity balm allowing for slow absorption over time.

Each of Lotion A, Lotion B, and Balm A comprise ingredients with anti-inflammatory and counter-irritant effects useful in the treatment of, e.g., pain and/or inflammation, especially as it relates to muscles and joints.

### Example 4: Non-Working Formulations

The following recipes were attempted in obtaining the compositions of the present disclosure but did not form stable emulsions for various reasons, e.g., thermal instability. Compositions were prepared following the general steps used in Example 3, except that the recipes varied.

TABLE 4
Non-working Lotion C

| | Percent | Mass |
|---|---|---|
| Ingredient | Composition | (mg) |
| Purified Water, Organic | 34.632% | 19740 |
| Ethyl Alcohol, Organic | 12.000% | 6840 |
| Olive Oil, Extra Virgin, Organic | 10.600% | 6042 |
| Sunflower Oil, Organic | 10.600% | 6042 |
| Hemp Seed Oil, Golden - Virgin, Organic | 10.600% | 6042 |
| Glycerin - USP, Organic | 4.678% | 2666.46 |
| Menthol Crystals, Organic | 3.000% | 1710 |
| Ravintsara (Camphor), Organic | 3.000% | 1710 |
| Eucalyptus Oil, Smithii, Organic | 3.000% | 1710 |
| Arnica, CO2 Extract Blend, Organic | 1.000% | 570 |
| Black Pepper Oil, Organic | 2.000% | 1140 |
| CBD Extract, Organic | 1.05% | 600 |
| Quillaja Saponin Extract, Organic | 1.000% | 570 |
| Spray Dried Guar Gum, Organic | 1.000% | 570 |
| Turmeric CO2 Extract- Organic | 1.000% | 570 |
| Sea Buckthorn Oil, Organic | 0.500% | 285 |
| Aloe Vera Powder, Organic | 0.337% | 192.3 |
| Total: | 100% | 57000 |

Lotion C was not thermostable and lost viscosity and broke emulsion after a period of time at somewhat elevated temperatures.

TABLE 5
Non-working lotion D

| | Percent | Mass |
|---|---|---|
| Ingredient | Composition | (mg) |
| Purified Water, Organic | 35.77% | 20387 |
| Ethyl Alcohol, Organic | 12.000% | 6840 |
| Olive Oil, Extra Virgin, Organic | 10.600% | 6042 |
| Sunflower Oil, Organic | 10.600% | 6042 |
| Hemp Seed Oil, Golden - Virgin, Organic | 10.600% | 6042 |
| Glycerin - USP, Organic | 4.680% | 2668 |
| Peppermint Oil, Piperita, Organic | 4.000% | 2280 |
| Arnica, CO2 Extract Blend, Organic | 1.000% | 570 |
| Lemon Balm Extract | 3.000% | 1710 |
| Eucalyptus Oil, Smithii, Organic | 2.000% | 1140 |
| Rose Absolute Oil | 1.100% | 627 |
| CBD Extract, Organic | 1.053% | 600 |
| Quillaja Saponin Extract, Organic | 1.00% | 570 |
| Chamomile Extract | 1.00% | 570 |
| Spray Dried Guar Gum, Organic | 1.00% | 570 |
| Aloe Vera Powder, Organic | 0.30% | 171 |
| Sea Buckthorn Oil, Organic | 0.30% | 171 |
| Total: | 100% | 57000 |

Lotion D was not thermostable and lost viscosity and broke emulsion after a period of time at somewhat elevated temperatures.

For Lotions C and D, when the emulsions broke, alcohol was observed to precipitate out. Lotion C and D had a thin, paint-like, free-flowing consistency with separated liquids and striations of white. FIG. 1A-E shows a comparison of working lotion embodiments A and B and non-working Lotions C and D. FIG. 1A shows Lotion A, which has a thicker consistency, retaining some of its shape when extruded from the lotion bottle. FIG. 1B shows Lotion B, which forms a distinct mound of thicker consistency lotion when extruded from the bottle. FIG. 1C shows Lotion C, which pours like a liquid from the container and shows clear separation, striations, and flocculation. FIG. 1D shows Lotion D, which pours like a liquid from the container and shows clear separation, striations, and flocculation. FIG. 1E shows a side by side comparison of all four lotions.

TABLE 6
Balm B

| | Percent | Mass |
|---|---|---|
| Ingredient | Composition | (mg) |
| Shea Nut Butter, White Refined, Organic | 33.50% | 9380 |
| Beeswax, Yellow, Organic | 27.00% | 7560 |
| MCT Oil, Unflavored, Organic | 26.929% | 7540.008 |
| Turmeric CO2 Extract, Organic | 4.00% | 1120 |
| CBD Extract, Organic | 3.57% | 999.992 |
| Arnica, CO2 Extract Blend, Organic | 2.00% | 560 |
| Menthol Crystals, Organic | 1.00% | 280 |
| Ravintsara (Camphor), Organic | 1.00% | 280 |
| Eucalyptus Oil, Smithii, Organic | 1.00% | 280 |
| Total: | 100% | 28000 |

Balm B formed a stable product, but had a problem of excessive sticking to the product lid that was resolved by the formulation of Balm A.

### Example 5: Additional Non-Working Lotion Formulations

Lotion formulation batches 15, 16A, 16B, 17A, 17B, and 21 did not form stable emulsions. Compositions were prepared following the general steps used in Examples 3 and 4, except that the recipes varied.

Formulation Batch 15, ingredients in Table 7.

TABLE 7
Formulation Batch 15

| % Composition | Amount Used (g) | Ingredient |
|---|---|---|
| 42.7674 | 214.5 | Water |
| 20 | 100.45 | Ethanol 200 proof |
| 5.6 | 27.92 | Olive Oil |
| 5.6 | 28.25 | Sunflower oil |
| 5.6 | 28.11 | hemp seed oil |
| 4.68 | 23.44 | Glycerin |
| 4 | 20.05 | Tea tree oil |
| 0.02 | 0.12 | Arnica |
| 0.98 | 4.92 | MCT |
| 3 | 15.2 | Lemon Balm Extract |
| 2 | 10.08 | Eucalyptus oil |
| 1.1 | 5.52 | Rose Geranium oil |
| 0.7 | 3.54 | CBD oil |
| 0.24 | 1.29 | Clove |
| 0.11 | 0.53 | Peppermint |
| 1 | 5 | Andean |
| 1 | 5.07 | Chamomile Extract |
| 1 | 5.01 | Sodium Alginate |
| 0.3 | 1.51 | Guar Gum |
| 0.3 | 1.64 | Sea Buckthorn Oil |

Formulation Batch 16A, ingredients in Table 8A.

TABLE 8A
Formulation Batch 16A

| % Composition | Amount Used (g) | Ingredient |
|---|---|---|
| 42.7674 | 214.65 | Water |
| 20 | 100.28 | Ethanol 200 proof |
| 5.6 | 28.12 | Olive Oil |
| 5.6 | 27.98 | Sunflower oil |
| 5.6 | 28.08 | Hemp seed oil |
| 4.68 | 23.39 | Glycerin |
| 4 | 20.03 | Tea tree oil |
| 0.02 | 0.12 | Arnica |
| 0.98 | 4.98 | MCT |
| 3 | 15.03 | Lemon Balm Extract |
| 2 | 10.03 | Eucalyptus oil |
| 1.1 | 5.57 | Rose Geranium oil |
| 0.73 | 3.73 | CBD oil |
| 0.24 | 1.26 | Clove |
| 0.1 | 0.52 | Peppermint |
| 1 | 5 | Andean |
| 1 | 5.09 | Chamomile Extract |
| 1 | 5 | Sodium Alginate |
| 0.3 | 1.54 | Guar Gum |
| 0.3 | 1.51 | Sea Buckthorn Oil |

Formulation Batch 16B, ingredients in Table 8B.

TABLE 8B
Formulation Batch 16B

| % Composition | Amount Used (g) | Ingredient |
|---|---|---|
| 42.7674 | 214.54 | Water |
| 20 | 99.81 | Ethanol 200 proof |
| 5.6 | 28.1 | Olive Oil |
| 5.6 | 28.02 | Sunflower oil |
| 5.6 | 28.05 | Hemp seed oil |
| 4.68 | 23.39 | Glycerin |
| 4 | 20.06 | Tea tree oil |
| 0.02 | 0.1 | Arnica |
| 0.98 | 4.89 | MCT |
| 3 | 15.22 | Lemon Balm Extract |
| 2 | 9.99 | Eucalyptus oil |
| 1.1 | 5.49 | Rose Geranium oil |
| 0.75 | 3.77 | CBD oil |
| 0.29 | 1.5 | Clove |
| 0.09 | 0.5 | Peppermint |
| 1 | 4.99 | Andean |
| 1 | 5 | Chamomile Extract |
| 1 | 5 | Sodium Alginate |
| 0.3 | 1.5 | Guar Gum |
| 0.3 | 1.5 | Sea Buckthorn Oil |

Formulation Batch 17A, ingredients in Table 9A.

TABLE 9A
Formulation Batch 17A

| % Composition | Amount Used (g) | Ingredient |
|---|---|---|
| 43.869 | 220.16 | Water |
| 20 | 100 | Ethanol 200 proof |
| 5.6 | 27.92 | Olive Oil |
| 5.6 | 27.94 | Sunflower oil |
| 0.6 | 3.1 | Hemp seed oil |
| 4.68 | 23.52 | Glycerin |
| 3 | 15.06 | Lavender |
| 3 | 15 | Menthol |
| 3 | 15.09 | Ravintsara |
| 3 | 15.11 | Eucalyptus |
| 0.02 | 0.1 | Arnica |
| 0.98 | 5.05 | MCT |
| 2 | 10.1 | Black Pepper |
| 0.74 | 3.72 | CBD |
| 0.29 | 1.5 | Clove |
| 0.1 | 0.55 | Peppermint |
| 1 | 5 | Andean |
| 1 | 5 | Sodium alginate |
| 1 | 5 | Turmeric |
| 0.3 | 1.45 | Sea buckthorn |
| 0.3 | 1.5 | Guar gum |

Formulation Batch 17B, ingredients in Table 9B.

TABLE 9B
Formulation Batch 17B

| % Composition | Amount Used (g) | Ingredient |
|---|---|---|
| 43.869 | 220.21 | Water |
| 20 | 100.08 | Ethanol 200 proof |
| 5.6 | 28.01 | Olive Oil |
| 5.6 | 28.02 | Sunflower oil |
| 0.6 | 3.03 | Hemp seed oil |
| 4.68 | 23.57 | Glycerin |
| 3 | 15.01 | Lavender |
| 3 | 15.05 | Menthol |
| 3 | 15.03 | Ravintsara |
| 3 | 15.18 | Eucalyptus |
| 0.02 | 0.11 | Arnica |
| 0.98 | 4.89 | MCT |
| 2 | 10.03 | Black Pepper |
| 0.74 | 3.72 | CBD |
| 0.3 | 1.52 | Clove |
| 0.09 | 0.49 | Peppermint |
| 1 | 5 | Andean |
| 1 | 5.01 | Sodium alginate |
| 1 | 5.05 | Turmeric |
| 0.3 | 1.55 | Sea buckthorn |
| 0.3 | 1.53 | Guar gum |

Formulation Batch 21, ingredients in Table 10.

TABLE 10
Formulation Batch 21

| % Composition | Amount Used (g) | Ingredient |
|---|---|---|
| 42.7674 | 214.51 | Water |
| 20 | 100.32 | Ethanol 200 proof |
| 5.6 | 28.02 | Olive Oil |
| 5.6 | 28.18 | Sunflower oil |
| 5.6 | 28.13 | Hemp seed oil |
| 4.68 | 23.54 | Glycerin |
| 3 | 15.03 | Tea tree oil |
| 0.02 | 0.1 | Arnica |
| 0.98 | 4.92 | MCT |
| 3 | 15.15 | Lemon Balm Extract |
| 2 | 10 | Eucalyptus oil |
| 1.1 | 5.6 | Rose Geranium oil |
| 1.0526 | 3.66 | CBD oil |
| 0.29 | 1.49 | Clove |
| 0.1 | 0.54 | Peppermint |
| 1 | 5.01 | Andean |
| 1 | 4.97 | Chamomile Extract |
| 1 | 5.03 | Sodium Alginate |
| 0.3 | 1.5 | Guar Gum |
| 0.3 | 1.59 | Sea Buckthorn Oil |
| 1 | 5 | Peppermint |

### INCORPORATION BY REFERENCE

The disclosures of U.S. Provisional Application No. 62/979,917 and U.S. Provisional Application No. 62/979,924 are incorporated by reference herein in their entireties. All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### ADDITIONAL EMBODIMENTS OF THE DISCLOSURE

Notwithstanding the appended claims, the disclosure sets forth the following embodiments:

A composition for topical application comprising greater than 95% organic ingredients.

A composition according to any one of the foregoing embodiments, wherein the composition is a topical oil-in-water emulsion composition.

A composition according to any one of the foregoing embodiments, wherein the composition is a lotion.

A composition according to any one of the foregoing embodiments, wherein the composition comprises sodium alginate.

A composition according to any one of the foregoing embodiments, wherein the composition comprises 0.1-5.0% w/w sodium alginate.

A composition according to any one of the foregoing embodiments, wherein the composition comprises 0.5-2.0% w/w sodium alginate.

A composition according to any one of the foregoing embodiments, wherein the composition comprises about 1.0% w/w sodium alginate.

A composition according to any one of the foregoing embodiments, wherein the composition has a viscosity greater than 1000 centipoise (cps).

A composition according to any one of the foregoing embodiments, wherein the composition has a viscosity greater than 2000 cps, greater than 3000 cps, greater than 4000 cps, or greater than 5000 cps.

A composition according to any one of the foregoing embodiments, wherein the composition has a viscosity in the range of 4000-6500 cps.

A composition according to any one of the foregoing embodiments, wherein the composition has a density in the range of 0.92-1.0 g/cm3

A composition according to any one of the foregoing embodiments, wherein the composition has a density of about 0.96 g/cm3.

A composition according to any one of the foregoing embodiments, wherein the water phase of the composition comprises 40-60% w/w of the composition.

A composition according to any one of the foregoing embodiments, wherein the water phase of the composition comprises 45-58% w/w of the composition.

A composition according to any one of the foregoing embodiments, wherein the oil phase of the composition comprises 15-30% w/w of the composition.

A composition according to any one of the foregoing embodiments, wherein the oil phase of the composition comprises 21-27% w/w of the composition.

A composition according to any one of the foregoing embodiments, wherein the alcohol phase of the composition comprises 15-30% w/w of the composition.

A composition according to any one of the foregoing embodiments, wherein the alcohol phase of the composition comprises 19-25% w/w of the composition.

A composition according to any one of the foregoing embodiments, wherein the composition is thermostable.

A composition according to any one of the foregoing embodiments, wherein the composition is stable at room temperature for a period of at least six months.

A composition according to any one of the foregoing embodiments, wherein the composition is produced by a process comprising the steps of:

a) Mixing the water phase ingredients together and then homogenizing;

b) Adding binders to the water phase ingredients and then homogenizing;

c) Adding the oil phase ingredients to the mixture of step (b) and then homogenizing;

d) Adding alcohol phase ingredients to the mixture of step (c) and then homogenizing.

A composition according to any one of the foregoing embodiments, wherein the composition is produced by a process in which the homogenization performed in each step is performed until the mixture is fully homogenized.

A composition according to any one of the foregoing embodiments, wherein the composition comprises a therapeutic oil blend as described herein.

### FURTHER NUMBERED EMBODIMENTS

1. A topical oil-in-water emulsion composition comprising: a) Water; b) Ethanol; c) Olive Oil; d) Sunflower Oil; e) Hemp Seed Oil; f) Glycerin; g) Lavender Oil; h) Menthol; i) Camphor; j) Eucalyptus Oil; k) Arnica Oil; l) Black Pepper Oil; m) CBD extract; n) Saponin; o) Sodium Alginate; p) Turmeric Oil; q) Sea Buckthorn Oil; and r) Guar Gum.
2. The composition of embodiment 1, wherein the composition comprises about: a) 40-60% Water; b) 10-30% Ethanol; c) 1-10% Olive Oil; d) 1-10% Sunflower Oil; e) 0.1-2.0% Hemp Seed Oil; f) 1-10% Glycerin; g) 0.1-5.0% Lavender Oil; h) 0.1-5.0% Menthol; i) 0.1-5.0% Camphor; j) 0.1-5.0% Eucalyptus Oil; k) 0.1-5.0% Arnica Oil; l) 0.1-5.0% Black Pepper Oil; m) 0.1-5.0% CBD extract; n) 0.1-5.0% Saponin; o) 0.1-5.0% Sodium Alginate; p) 0.1-5.0% Turmeric Oil; q) 0.1-1.0% Sea Buckthorn Oil; and r) 0.1-1.0% Guar Gum, wherein the percent content is weight of ingredient by weight of the composition.
3. The composition of embodiment 1, wherein the composition comprises about: a) 51% Water; b) 20% Ethanol; c) 6% Olive Oil; d) 6% Sunflower Oil; e) 1% Hemp Seed Oil; f) 5% Glycerin; g) 1.5% Lavender Oil; h) 1.5% Menthol; i) 1.5% Camphor; j) 1.5% Eucalyptus Oil; k) 1% Arnica Oil; l) 1% Black Pepper Oil; m) 1% CBD extract; n) 1% Saponin; o) 1% Sodium Alginate; p) 1% Turmeric Oil; q) 0.3% Sea Buckthorn Oil; and r) 0.3% Guar Gum, wherein the percent content is weight of ingredient by weight of the composition.
4. The composition of embodiment 1, wherein the composition comprises per two ounce volume about: a) 20-40 g Water; b) 5-20 g Ethanol; c) 1-5 g Olive Oil; d) 1-5 g Sunflower Oil; e) 0.1-0.5 g Hemp Seed Oil; f) 1-5 g Glycerin; g) 0.1-2 g Lavender Oil; h) 0.1-2 g Menthol; i) 0.1-2 g Camphor; j) 0.1-2 g Eucalyptus Oil; k) 0.1-2 g Arnica Oil; l) 0.1-2 g Black Pepper Oil; m) 0.1-2 g CBD extract; n) 0.1-2 g Saponin; o) 0.1-2 g Sodium Alginate; p) 0.1-2 g Turmeric Oil; q) 0.1-2 g Sea Buckthorn Oil; and r) 0.1-2 g Guar Gum.
5. The composition of embodiment 1, wherein the composition comprises per two ounce volume about: a) 29 g Water; b) 11 g Ethanol; c) 3 g Olive Oil; d) 3 g Sunflower Oil; e) 0.3 g Hemp Seed Oil; f) 2.5 g Glycerin; g) 0.9 g Lavender Oil; h) 0.9 g Menthol; i) 0.9 g Camphor; j) 0.9 g Eucalyptus Oil; k) 0.6 g Arnica Oil; l) 0.6 g Black Pepper Oil; m) 0.6 g CBD extract; n) 0.6 g Saponin; o) 0.6 g Sodium Alginate; p) 0.6 g Turmeric Oil; q) 0.2 g Sea Buckthorn Oil; and r) 0.2 g Guar Gum.
6. A topical oil-in-water emulsion composition comprising: a) Water; b) Ethanol; c) Olive Oil; d) Sunflower Oil; e) Hemp Seed Oil; f) Glycerin; g) Tea Tree Oil; h) Arnica Oil; i) Lemon Balm Extract; j) Eucalyptus Oil; k) Rose Geranium Oil; l) CBD extract; m) Saponin; n) Chamomile Extract; o) Sodium Alginate; p) Guar Gum; q) Sea Buckthorn Oil; and r) Peppermint Oil.
7. The composition of embodiment 6, wherein the composition comprises about: a) 40-50% Water; b) 10-30% Ethanol; c) 1-10% Olive Oil; d) 1-10% Sunflower Oil; e) 1-10% Hemp Seed Oil; f) 1-10% Glycerin; g) 0.1-5% Tea Tree Oil; h) 0.1-5% Arnica Oil; i) 0.1-5% Lemon Balm Extract; j) 0.1-5% Eucalyptus Oil; k) 0.1-5% Rose Geranium Oil; l) 0.1-5% CBD extract; m) 0.1-5% Saponin; n) 0.1-5% Chamomile Extract; o) 0.1-5% Sodium Alginate; p) 0.1-1% Guar Gum; q) 0.1-1% Sea Buckthorn Oil; and r) 0.1-5% Peppermint Oil, wherein the percent content is weight of ingredient by weight of the composition.
8. The composition of embodiment 6, wherein the composition comprises about: a) 43% Water; b) 20% Ethanol; c) 6% Olive Oil; d) 6% Sunflower Oil; e) 6% Hemp Seed Oil; f) 5% Glycerin; g) 1% Tea Tree Oil; h) 1% Arnica Oil; i) 3% Lemon Balm Extract; j) 2% Eucalyptus Oil; k) 1% Rose Geranium Oil; l) 1% CBD extract; m) 1% Saponin; n) 1% Chamomile Extract; o) 1% Sodium Alginate; p) 0.3% Guar Gum; q) 0.3% Sea Buckthorn Oil; and r) 3% Peppermint Oil, wherein the percent content is weight of ingredient by weight of the composition.
9. The composition of embodiment 6, wherein the composition comprises per two ounce volume about: a) 20-30 g Water; b) 5-15 g Ethanol; c) 1-5 g Olive Oil; d) 1-5 g Sunflower Oil; e) 1-5 g Hemp Seed Oil; f) 1-5 g Glycerin; g) 0.1-2.0 g Tea Tree Oil; h) 0.1-2.0 g Arnica Oil; i) 0.1-5.0 g Lemon Balm Extract; j) 0.1-5.0 g Eucalyptus Oil; k) 0.1-2.0 g Rose Geranium Oil; l) 0.1-2.0 g CBD extract; m) 0.1-2.0 g Saponin; n) 0.1-2.0 g Chamomile Extract; o) 0.1-2.0 g Sodium Alginate; p) 0.1-1.0 g Guar Gum; q) 0.1-1.0 g Sea Buckthorn Oil; and r) 1-5 g Peppermint Oil.
10. The composition of embodiment 6, wherein the composition comprises per two ounce volume about: a) 24 g Water; b) 11 g Ethanol; c) 3 g Olive Oil; d) 3 g Sunflower Oil; e) 3 g Hemp Seed Oil; f) 3 g Glycerin; g) 0.6 g Tea Tree Oil; h) 0.6 g Arnica Oil; i) 2 g Lemon Balm Extract; j) 1 g Eucalyptus Oil; k) 0.6 g Rose Geranium Oil; l) 0.6 g CBD extract; m) 0.6 g Saponin; n) 0.6 g Chamomile Extract; o) 0.6 g Sodium Alginate; p) 0.2 g Guar Gum; q) 0.2 g Sea Buckthorn Oil; and r) 2 g Peppermint Oil.
11. A balm composition comprising: a) Shea Nut Butter; b) Beeswax; c) MCT Oil; d) Turmeric Oil; e) CBD extract; f) Arnica Oil; g) Menthol; h) Camphor; and i) Eucalyptus Oil.
12. The composition of embodiment 11, wherein the composition comprises about: a) 10-40% Shea Nut Butter; b) 20-50% Beeswax; c) 20-30% MCT Oil; d) 1-10% Turmeric Oil; e) 1-10% CBD extract; f) 1-5% Arnica Oil; g) 0.1-5.0% Menthol; h) 0.1-5.0% Camphor; and i) 0.1-5.0% Eucalyptus Oil, wherein the percent content is weight of ingredient by weight of the composition.
13. The composition of embodiment 11, wherein the composition comprises about: a) 30% Shea Nut Butter; b) 30% Beeswax; c) 27% MCT Oil; d) 4% Turmeric Oil; e) 4% CBD extract; f) 2% Arnica Oil; g) 1% Menthol; h) 1% Camphor; and i) 1% Eucalyptus Oil, wherein the percent content is weight of ingredient by weight of the composition.
14. The composition of embodiment 11, wherein the composition comprises per one ounce about: a) 5-15 g Shea Nut Butter; b) 5-15 g Beeswax; c) 5-10 g MCT Oil; d) 0.1-5 g Turmeric Oil; e) 0.1-5 g CBD extract; f) 0.1-5 g Arnica Oil; g) 0.1-2 g Menthol; h) 0.1-2 g Camphor; and i) 0.1-2 g Eucalyptus Oil.
15. The composition of embodiment 11, wherein the composition comprises per one ounce about: a) 9 g Shea Nut Butter; b) 8 g Beeswax; c) 7.5 g MCT Oil; d) 1 g Turmeric Oil; e) 1 g CBD extract; f) 0.5 g Arnica Oil; g) 0.3 g Menthol; h) 0.3 g Camphor; and i) 0.3 g Eucalyptus Oil.
16. An oil-in-water emulsion composition comprising:
a) 40-60% water by weight;
b) 11%-30% carrier oil by weight;
c) at least 1% cannabinoid by weight;
d) a foaming agent; and
e) an emulsion stabilizer;
wherein percent content is by weight of the topical oil-in-water emulsion composition, and wherein the oil-in-water composition is thermostable.
17. The oil-in-water emulsion composition of embodiment 16, comprising an essential oil selected from the group consisting of: rose oil, menthol, tea tree oil, lavender, camphor, arnica, black pepper oil, turmeric, peppermint, eucalyptus oil, lemon balm, chamomile oil, clove oil, rose geranium oil, and sea buckthorn oil.
18. The oil-in-water emulsion composition of embodiment 16, wherein the foaming agent comprises saponin.
19. The oil-in-water emulsion composition of embodiment 16, wherein the foaming agent comprises 0.1-5.0% saponin by weight.
20. The oil-in-water emulsion composition of embodiment 16, wherein the emulsion stabilizer comprises sodium alginate or sodium alginate and Guar Gum.
21. The oil-in-water emulsion composition of embodiment 16, wherein the emulsion stabilizer comprises 0.1-5.0% sodium alginate by weight.
22. The oil-in-water emulsion composition of embodiment 16, wherein the cannabinoid is comprised in a Therapeutic Oil Blend.
23. The oil-in-water emulsion composition of embodiment 16, wherein the cannabinoid comprises cannabidiol.
24. The oil-in-water emulsion composition of embodiment 16, comprising ethanol.
25. The oil-in-water emulsion composition of embodiment 16, comprising 10-30% ethanol by weight.
26. The oil-in-water emulsion composition of embodiment 16, comprising glycerin by weight.
27. The oil-in-water emulsion composition of embodiment 16, comprising 1-10% glycerin by weight.
28. The oil-in-water emulsion composition of embodiment 16, wherein the oil-in-water emulsion composition has reduced or no: separation, discoloration, or change in consistency at 37° C. for a period of three months.
29. The oil-in-water emulsion composition of embodiment 16, wherein the thermostability is determined by evaluating at least one of: separation, discoloration, or consistency, of the oil-in-water emulsion composition at 45° C. for a period of three months.
30. The oil-in-water emulsion composition of embodiment 16, wherein the oil-in-water emulsion is composed of at least 95% USDA certified organic ingredients.
31. The oil-in-water emulsion composition of embodiment 16, wherein the oil-in-water emulsion exhibits a viscosity of about 3,000-7,000 cps.
32. An oil-in-water emulsion composition, that comprises:

### a) Water;

### b) Ethanol;

### c) Olive Oil;

### d) Sunflower Oil;

### e) Hemp Seed Oil;

### f) Glycerin;

### g) Lavender Oil;

### h) Menthol;

### i) Camphor;

### j) Eucalyptus Oil;

### k) Arnica Oil;

### l) Black Pepper Oil;

### m) Cannabinoid;

### n) Saponin;

### o) Sodium Alginate;

### p) Turmeric Oil;

### q) Sea Buckthorn Oil; and

### r) Guar Gum.

33. The oil-in-water emulsion composition of embodiment 32, wherein the oil-in-water emulsion composition comprises:
a) 40-60% Water by weight;
b) 10-30% Ethanol by weight;
c) 1-10% Olive Oil by weight;
d) 1-10% Sunflower Oil by weight;
e) 0.1-2.0% Hemp Seed Oil by weight;
f) 1-10% Glycerin by weight;
g) 0.1-5.0% Lavender Oil by weight;
h) 0.1-5.0% Menthol by weight;
i) 0.1-5.0% Camphor by weight;
j) 0.1-5.0% Eucalyptus Oil by weight;
k) 0.1-5.0% Arnica Oil by weight;
l) 0.1-5.0% Black Pepper Oil by weight;
m) 0.1-5.0% Cannabinoid by weight;
n) 0.1-5.0% Saponin by weight;
o) 0.1-5.0% Sodium Alginate by weight;
p) 0.1-5.0% Turmeric Oil by weight;
q) 0.1-1.0% Sea Buckthorn Oil by weight; and
r) 0.1-1.0% Guar Gum by weight,
wherein percent content is by weight of the oil-in-water emulsion composition.
34. The oil-in-water emulsion composition of embodiment 32, wherein the oil-in-water emulsion composition comprises about:
a) 51% Water by weight;
b) 20% Ethanol by weight;
c) 6% Olive Oil by weight;
d) 6% Sunflower Oil by weight;
e) 1% Hemp Seed Oil by weight;
f) 5% Glycerin by weight;
g) 1.5% Lavender Oil by weight;
h) 1.5% Menthol by weight;
i) 1.5% Camphor by weight;
j) 1.5% Eucalyptus Oil by weight;
k) 1% Arnica Oil by weight;
l) 1% Black Pepper Oil by weight;
m) 1% Cannabinoid by weight;
n) 1% Saponin by weight;
o) 1% Sodium Alginate by weight;
p) 1% Turmeric Oil by weight;
q) 0.3% Sea Buckthorn Oil by weight; and

### r) 0.3% Guar Gum,

wherein percent content is by weight of the oil-in-water emulsion composition.
35. An oil-in-water emulsion composition, that comprises:

### a) Water;

### b) Ethanol;

### c) Olive Oil;

### d) Sunflower Oil;

### e) Hemp Seed Oil;

### f) Glycerin;

### g) Tea Tree Oil;

### h) Arnica Oil;

### i) Lemon Balm Oil;

### j) Eucalyptus Oil;

### k) Rose Geranium Oil;

### l) Cannabinoid;

### m) Saponin;

### n) Chamomile Oil;

### o) Sodium Alginate;

### p) Guar Gum;

### q) Sea Buckthorn Oil; and

### r) Peppermint Oil.

36. The oil-in-water emulsion composition of embodiment 35, wherein the oil-in-water emulsion composition comprises:
a) 40-50% Water by weight;
b) 10-30% Ethanol by weight;
c) 1-10% Olive Oil by weight;
d) 1-10% Sunflower Oil by weight;
e) 1-10% Hemp Seed Oil by weight;
f) 1-10% Glycerin by weight;
g) 0.1-5% Tea Tree Oil by weight;
h) 0.1-5% Arnica Oil by weight;
i) 0.1-5% Lemon Balm Oil by weight;
j) 0.1-5% Eucalyptus Oil by weight;
k) 0.1-5% Rose Geranium Oil by weight;
l) 0.1-5% Cannabinoid by weight;
m) 0.1-5% Saponin by weight;
n) 0.1-5% Chamomile Oil by weight;
o) 0.1-5% Sodium Alginate by weight;
p) 0.1-1% Guar Gum by weight;
q) 0.1-1% Sea Buckthorn Oil by weight; and
r) 0.1-5% Peppermint Oil by weight,
wherein percent content is by weight of the oil-in-water emulsion composition.
37. The oil-in-water emulsion composition of embodiment 35, wherein the oil-in-water emulsion composition comprises about:
a) 43% Water by weight;
b) 20% Ethanol by weight;
c) 6% Olive Oil by weight;
d) 6% Sunflower Oil by weight;
e) 6% Hemp Seed Oil by weight;
f) 5% Glycerin by weight;
g) 1% Tea Tree Oil by weight;
h) 1% Arnica Oil by weight;
i) 3% Lemon Balm Oil by weight;
j) 2% Eucalyptus Oil by weight;
k) 1% Rose Geranium Oil by weight;
l) 1% Cannabinoid by weight;
m) 1% Saponin by weight;
n) 1% Chamomile Oil by weight;
o) 1% Sodium Alginate by weight;
p) 0.3% Guar Gum by weight;
q) 0.3% Sea Buckthorn Oil by weight; and
r) 3% Peppermint Oil by weight,
wherein the percent content is by weight of the oil-in-water emulsion composition.
38. A balm composition, that comprises:

### a) Shea Nut Butter;

### b) Beeswax;

### c) MCT Oil;

### d) Turmeric Oil;

### e) Cannabinoid;

### f) Arnica Oil;

### g) Menthol;

### h) Camphor; and

### i) Eucalyptus Oil.

39. The balm composition of embodiment 38, wherein the balm composition comprises:
a) 10-40% Shea Nut Butter by weight;
b) 20-50% Beeswax by weight;
c) 20-30% MCT Oil by weight;
d) 1-10% Turmeric Oil by weight;
e) 1-10% Cannabidiol by weight;
f) 1-5% Arnica Oil by weight;
g) 0.1-5.0% Menthol by weight;
h) 0.1-5.0% Camphor by weight; and
i) 0.1-5.0% Eucalyptus Oil by weight,
wherein percent content is by weight of the balm composition.
40. The balm composition of embodiment 38, wherein the balm composition comprises about:
a) 30% Shea Nut Butter by weight;
b) 30% Beeswax by weight;
c) 27% MCT Oil by weight;
d) 4% Turmeric Oil by weight;
e) 4% Cannabidiol by weight;
f) 2% Arnica Oil by weight;
g) 1% Menthol by weight;
h) 1% Camphor by weight; and
i) 1% Eucalyptus Oil by weight,
wherein percent content is by weight of the balm composition.
41. The composition of any one of embodiments 1-40, comprising guar gum.

1. An oil-in-water emulsion composition comprising:
a) 40-60% water by weight;
b) 11%-30% carrier oil by weight;
c) at least 1% cannabinoid by weight;
d) a foaming agent; and
e) an emulsion stabilizer;
wherein percent content is by weight of the topical oil-in-water emulsion composition, and wherein the oil-in-water composition is thermostable.

2. The oil-in-water emulsion composition of claim 1, comprising an essential oil selected from the group consisting of: rose oil, menthol, tea tree oil, lavender, camphor, arnica, black pepper oil, turmeric, peppermint, eucalyptus oil, lemon balm, chamomile oil, clove oil, rose geranium oil, and sea buckthorn oil.

3. The oil-in-water emulsion composition of claim 1, wherein the foaming agent comprises saponin.

4. The oil-in-water emulsion composition of claim 1, wherein the foaming agent comprises 0.1-5.0% saponin by weight.

5. The oil-in-water emulsion composition of claim 1, wherein the emulsion stabilizer comprises sodium alginate or sodium alginate and Guar Gum.

6. The oil-in-water emulsion composition of claim 1, wherein the emulsion stabilizer comprises 0.1-5.0% sodium alginate by weight.

7. The oil-in-water emulsion composition of claim 1, wherein the cannabinoid is comprised in a Therapeutic Oil Blend.

8. The oil-in-water emulsion composition of claim 1, wherein the cannabinoid comprises cannabidiol.

9. The oil-in-water emulsion composition of claim 1, comprising ethanol.

10. The oil-in-water emulsion composition of claim 1, comprising 10-30% ethanol by weight.

11. The oil-in-water emulsion composition of claim 1, comprising glycerin by weight.

12. The oil-in-water emulsion composition of claim 1, comprising 1-10% glycerin by weight.

13. The oil-in-water emulsion composition of claim 1, wherein the oil-in-water emulsion composition has reduced or no: separation, discoloration, or change in consistency at 37° C. for a period of three months.

14. The oil-in-water emulsion composition of claim 1, wherein the thermostability is determined by evaluating at least one of: separation, discoloration, or consistency, of the oil-in-water emulsion composition at 45° C. for a period of three months.

15. The oil-in-water emulsion composition of claim 1, wherein the oil-in-water emulsion is composed of at least 95% USDA certified organic ingredients.

16. The oil-in-water emulsion composition of claim 1, wherein the oil-in-water emulsion exhibits a viscosity of about 3,000-7,000 cps.

17. An oil-in-water emulsion composition, that comprises:
a) Water;
b) Ethanol;
c) Olive Oil;
d) Sunflower Oil;
e) Hemp Seed Oil;
f) Glycerin;
g) Lavender Oil;
h) Menthol;
i) Camphor;
j) Eucalyptus Oil;
k) Arnica Oil;
l) Black Pepper Oil;
m) Cannabinoid;
n) Saponin;
o) Sodium Alginate;
p) Turmeric Oil;
q) Sea Buckthorn Oil; and
r) Guar Gum.

18. The oil-in-water emulsion composition of claim 17, wherein the oil-in-water emulsion composition comprises:
a) 40-60% Water by weight;
b) 10-30% Ethanol by weight;
c) 1-10% Olive Oil by weight;
d) 1-10% Sunflower Oil by weight;
e) 0.1-2.0% Hemp Seed Oil by weight;
f) 1-10% Glycerin by weight;
g) 0.1-5.0% Lavender Oil by weight;
h) 0.1-5.0% Menthol by weight;
i) 0.1-5.0% Camphor by weight;
j) 0.1-5.0% Eucalyptus Oil by weight;
k) 0.1-5.0% Arnica Oil by weight;
l) 0.1-5.0% Black Pepper Oil by weight;
m) 0.1-5.0% Cannabinoid by weight;
n) 0.1-5.0% Saponin by weight;
o) 0.1-5.0% Sodium Alginate by weight;
p) 0.1-5.0% Turmeric Oil by weight;
q) 0.1-1.0% Sea Buckthorn Oil by weight; and
r) 0.1-1.0% Guar Gum by weight,
wherein percent content is by weight of the oil-in-water emulsion composition.

19. The oil-in-water emulsion composition of claim 17, wherein the oil-in-water emulsion composition comprises about:
a) 51% Water by weight;
b) 20% Ethanol by weight;
c) 6% Olive Oil by weight;
d) 6% Sunflower Oil by weight;
e) 1% Hemp Seed Oil by weight;
f) 5% Glycerin by weight;
g) 1.5% Lavender Oil by weight;
h) 1.5% Menthol by weight;
i) 1.5% Camphor by weight;
j) 1.5% Eucalyptus Oil by weight;
k) 1% Arnica Oil by weight;
l) 1% Black Pepper Oil by weight;
m) 1% Cannabinoid by weight;
n) 1% Saponin by weight;
o) 1% Sodium Alginate by weight;
p) 1% Turmeric Oil by weight;
q) 0.3% Sea Buckthorn Oil by weight; and
r) 0.3% Guar Gum,
wherein percent content is by weight of the oil-in-water emulsion composition.

20. An oil-in-water emulsion composition, that comprises:
a) Water;
b) Ethanol;
c) Olive Oil;
d) Sunflower Oil;
e) Hemp Seed Oil;
f) Glycerin;
g) Tea Tree Oil;
h) Arnica Oil;
i) Lemon Balm Oil;
j) Eucalyptus Oil;
k) Rose Geranium Oil;
l) Cannabinoid;
m) Saponin;
n) Chamomile Oil;
o) Sodium Alginate;
p) Guar Gum;
q) Sea Buckthorn Oil; and
r) Peppermint Oil.

21. The oil-in-water emulsion composition of claim 20, wherein the oil-in-water emulsion composition comprises:
a) 40-50% Water by weight;
b) 10-30% Ethanol by weight;
c) 1-10% Olive Oil by weight;
d) 1-10% Sunflower Oil by weight;
e) 1-10% Hemp Seed Oil by weight;
f) 1-10% Glycerin by weight;
g) 0.1-5% Tea Tree Oil by weight;
h) 0.1-5% Arnica Oil by weight;
i) 0.1-5% Lemon Balm Oil by weight;
j) 0.1-5% Eucalyptus Oil by weight;
k) 0.1-5% Rose Geranium Oil by weight;
l) 0.1-5% Cannabinoid by weight;
m) 0.1-5% Saponin by weight;
n) 0.1-5% Chamomile Oil by weight;
o) 0.1-5% Sodium Alginate by weight;
p) 0.1-1% Guar Gum by weight;
q) 0.1-1% Sea Buckthorn Oil by weight; and
r) 0.1-5% Peppermint Oil by weight,
wherein percent content is by weight of the oil-in-water emulsion composition.

22. The oil-in-water emulsion composition of claim 20, wherein the oil-in-water emulsion composition comprises about:
a) 43% Water by weight;
b) 20% Ethanol by weight;
c) 6% Olive Oil by weight;
d) 6% Sunflower Oil by weight;
e) 6% Hemp Seed Oil by weight;
f) 5% Glycerin by weight;
g) 1% Tea Tree Oil by weight;
h) 1% Arnica Oil by weight;
i) 3% Lemon Balm Oil by weight;
j) 2% Eucalyptus Oil by weight;
k) 1% Rose Geranium Oil by weight;
l) 1% Cannabinoid by weight;
m) 1% Saponin by weight;
n) 1% Chamomile Oil by weight;
o) 1% Sodium Alginate by weight;
p) 0.3% Guar Gum by weight;
q) 0.3% Sea Buckthorn Oil by weight; and
r) 3% Peppermint Oil by weight,
wherein the percent content is by weight of the oil-in-water emulsion composition.

23. A balm composition, that comprises:
a) Shea Nut Butter;
b) Beeswax;
c) MCT Oil;
d) Turmeric Oil;
e) Cannabinoid;
f) Arnica Oil;
g) Menthol;
h) Camphor; and
i) Eucalyptus Oil.

24. The balm composition of claim 23, wherein the balm composition comprises:
a) 10-40% Shea Nut Butter by weight;
b) 20-50% Beeswax by weight;
c) 20-30% MCT Oil by weight;
d) 1-10% Turmeric Oil by weight;
e) 1-10% Cannabidiol by weight;
f) 1-5% Arnica Oil by weight;
g) 0.1-5.0% Menthol by weight;
h) 0.1-5.0% Camphor by weight; and
i) 0.1-5.0% Eucalyptus Oil by weight,
wherein percent content is by weight of the balm composition.

25. The balm composition of claim 23, wherein the balm composition comprises about:
a) 30% Shea Nut Butter by weight;
b) 30% Beeswax by weight;
c) 27% MCT Oil by weight;
d) 4% Turmeric Oil by weight;
e) 4% Cannabidiol by weight;
f) 2% Arnica Oil by weight;
g) 1% Menthol by weight;
h) 1% Camphor by weight; and
i) 1% Eucalyptus Oil by weight,
wherein percent content is by weight of the balm composition.
